(19) United States
(12) Patent Application Publication
(10) Pub. No.: US 2023/0183798 A1
(43) Pub. Date: Jun. 15, 2023

(54) COMPOSITIONS AND METHODS FOR MODIFYING POLYMERASE-NUCLEIC ACID COMPLEXES

(71) Applicant: Pacific Biosciences of California, Inc., Menlo Park, CA (US)

(72) Inventors: Hari K.K. Subramanian, San Diego, CA (US); Aaron W. Feldman, San Diego, CA (US); Chih-Yuan Chen, San Diego, CA (US); Denis Malyshev, San Diego, CA (US); Greg Richmond, San Diego, CA (US)

(21) Appl. No.: 17/923,506

(22) PCT Filed: Apr. 30, 2021
(86) PCT No.: PCT/US2021/030146

§ 371 Date: Nov. 4, 2022

Related U.S. Application Data
(60) Provisional application No. 63/020,115, filed on May 5, 2020.

(51) Int. Cl. C12Q 1/6874

(52) U.S. Cl. CPC C12Q 1/6874

(57) ABSTRACT

Provided herein include a method for modifying polymerase-nucleic acid complexes, including (a) providing a plurality of surface-immobilized polymerase-nucleic acid complexes in a vessel, wherein the nucleic acid includes a primed-template nucleic acid, wherein at least a subset of the surface-immobilized polymerase-nucleic acid complexes include ternary complexes further including nucleotides; and (b) washing the surface with an aqueous solution including a diol, sulfoxide or polyol, thereby removing the nucleotides from the vessel and retaining the surface-immobilized polymerase-nucleic acid complexes in the vessel.

### RELATED APPLICATIONS

The present application is a U.S. national phase application under 35 U.S.C. § 371 of

International Application No. PCT/US2021/030146, filed on Apr. 30, 2021 and published as WO 2021/225886 A1 Nov. 11, 2021, which claims priority to U.S. Provisional Application No. 63/020,115, filed May 5, 2020; the content of each of which is incorporated herein by reference in its entirety.

### BACKGROUND

The present disclosure relates generally to detection of nucleic acids and has specific applicability to nucleic acid sequencing technology.

Accurate sequence determination of a template nucleic acid strand is important for molecular diagnostics. Identification of a single nucleotide base from among alternatives at a known position can serve as the basis for analysis of single nucleotide polymorphisms (SNPs). A SNP can in turn be used to determine a phenotype for the individual such as susceptibility to a disease or propensity for having a desirable trait. Detecting genetic variants in a patient can provide an indication of the efficacy for certain medications to treat the patient or the risk of adverse side effects when treating the patient with certain medications.

Commercially available nucleic acid sequencing platforms have vastly increased our knowledge of the genetic underpinnings of actionable traits. Improvements in sequencing biochemistry and detection hardware continue. However, many platforms have achieved only relatively short reads and errors in the reads are a perennial difficulty. Massively parallel processing allows many short reads to be obtained and then knitted together to assemble a larger genomic sequence. The number of reads can be increased to achieve improved accuracy. For example, millions of reads that are each only a couple of hundred nucleotides in length can be assembled together to arrive at a human genome that is about 3 billion nucleotides long. The time and resources required to achieve massively parallel processing of the DNA and high throughput assembly of the data would be alleviated by increasing the length and the accuracy of the sequencing reads. The present invention addresses this need and provides related advantages as well.

### BRIEF SUMMARY

The present disclosure provides a composition, including a polymerase-nucleic acid complex, wherein the nucleic acid includes a primed-template nucleic acid in contact with an aqueous solution, wherein the aqueous solution includes a polyol, diol, sulfone or sulfoxide.

The present disclosure provides a method for modifying polymerase-nucleic acid complexes. The method can, for example, include the step of (a) providing a plurality of polymerase-nucleic acid complexes each comprises a polymerase and a primed-template nucleic acid, wherein at least a subset of the polymerase-nucleic acid complexes are ternary complexes further comprising nucleotides; and (b) contacting the plurality of polymerase-nucleic acid complexes with an aqueous solution comprising a polyol, alcohol, aliphatic diol, sulfone, sulfoxide, or a combination thereof, thereby dissociating the nucleotides from the subset of polymerase-nucleic acid complexes. The aqueous solution can further comprise additional components, such as Lithium, Betaine, or both. The plurality of polymerase-nucleic acid complexes can be immobilized on a surface, present in a vessel, or both. The method can include the steps of (a) providing a plurality of surface-immobilized polymerase-nucleic acid complexes in a vessel, wherein the nucleic acid includes a primed-template nucleic acid, wherein at least a subset of the surface-immobilized polymerase-nucleic acid complexes includes ternary complexes further comprising nucleotides; and (b) washing the surface with an aqueous solution comprising a polyol, diol, sulfone or sulfoxide, thereby removing the nucleotides from the vessel and retaining the surface-immobilized polymerase-nucleic acid complexes in the vessel.

A method for modifying polymerase-nucleic acid complexes can include the steps of (a) contacting a plurality of polymerases with a plurality of primed-template nucleic acids and with a plurality of nucleotides, thereby providing a plurality of surface-immobilized polymerase-nucleic acid complexes in the vessel, each of the surface-immobilized polymerase-nucleic acid complexes including a polymerase of the plurality of polymerases and a primed-template nucleic acid of the plurality of primed-template nucleic acids, the nucleotides in the ternary complexes including nucleotides of the plurality of nucleotides; and (b) washing the surface with an aqueous solution comprising a polyol, diol, sulfone or sulfoxide, thereby removing the nucleotides from the vessel and retaining the surface-immobilized polymerase-nucleic acid complexes in the vessel.

A method for modifying polymerase-nucleic acid complexes can include the steps of (a) providing a plurality of surface-immobilized polymerase-nucleic acid complexes in a vessel, wherein the nucleic acid includes a primed-template nucleic acid, wherein at least a subset of the surface-immobilized polymerase-nucleic acid complexes includes ternary complexes further comprising nucleotides; (b) washing the surface with an aqueous solution comprising a polyol, diol, sulfone or sulfoxide, thereby removing the nucleotides from the vessel and retaining the surface-immobilized polymerase-nucleic acid complexes in the vessel; and (c) delivering a solution including a plurality of second nucleotides to the vessel, whereby at least a second subset of the surface-immobilized polymerase-nucleic acid complexes includes ternary complexes further including second nucleotides from the plurality of second nucleotides.

This disclosure further provides a method for identifying a nucleotide in a primed-template nucleic acid. The method can include the steps of (a) providing a vessel having a primed-template nucleic acid, polymerase and a nucleotide cognate of a first base type; (b) examining the vessel for a stabilized ternary complex including the polymerase and the nucleotide cognate of the first base type bound at a base position of the primed-template nucleic acid; (c) washing the vessel with an aqueous solution including a polyol, diol, sulfone or sulfoxide, thereby removing the nucleotides from the vessel and retaining the primed-template nucleic acid and the polymerase from step (b); (d) delivering a nucleotide cognate of a second base type to the vessel after step (c); (e) examining the vessel for a stabilized ternary complex including the polymerase and the nucleotide cognate of the second base type bound at the base position of the primed-template nucleic acid; and (f) identifying the type of nucleotide at the base position of the primed-template nucleic acid. Optionally, the method can further include steps of (g) adding a nucleotide to the primer of the primed-template nucleic acid, whereby the vessel includes an extended primed-template nucleic acid; (h) delivering a second polymerase and a nucleotide cognate of the first base type to the vessel; and (i) repeating steps (b) through (f) using the extended primed-template instead of the primed-template nucleic acid and using the second polymerase instead of the polymerase.

In some embodiments, a method for identifying a nucleotide in a primed-template nucleic acid can include the steps of (a) providing a vessel having a primed-template nucleic acid, polymerase and a nucleotide cognate of a first base type; (b) examining the vessel for a stabilized ternary complex including the polymerase and the nucleotide cognate of the first base type bound at a base position of the primed-template nucleic acid; (c) washing the vessel with an aqueous solution including a polyol, diol, sulfone or sulfoxide, thereby removing the nucleotides from the vessel and retaining the primed-template nucleic acid and the polymerase from step (b); (d) delivering a nucleotide cognate of a second base type to the vessel after step (c); (e) examining the vessel for a stabilized ternary complex including the polymerase and the nucleotide cognate of the second base type bound at the base position of the primed-template nucleic acid; (f) identifying the type of nucleotide at the base position of the primed-template nucleic acid; (g) delivering a nucleotide cognate of a third base type to the vessel, whereby the vessel retains the primed-template nucleic acid and the polymerase from step (b); and (h) examining the vessel for a stabilized ternary complex having the polymerase and the nucleotide cognate of the third base type bound at the base position of the primed-template nucleic acid. Optionally, the method can further include steps of (i) adding a nucleotide to the primer of the primed-template nucleic acid, whereby the vessel includes an extended primed-template nucleic acid; (j) delivering a second polymerase and a nucleotide cognate of the first base type to the vessel; and (k) repeating steps (b) through (h) using the extended primed-template instead of the primed-template nucleic acid and using the second polymerase instead of the polymerase.

In some embodiments, a method for identifying a nucleotide in a primed-template nucleic acid can include the steps of (a) providing a vessel having a primed-template nucleic acid, polymerase and a nucleotide cognate of a first base type; (b) examining the vessel for a stabilized ternary complex including the polymerase and the nucleotide cognate of the first base type bound at a base position of the primed-template nucleic acid; (c) washing the vessel with an aqueous solution including a polyol, diol, sulfone or sulfoxide, thereby removing the nucleotides from the vessel and retaining the primed-template nucleic acid and the polymerase from step (b); (d) delivering a nucleotide cognate of a second base type to the vessel after step (c); (e) examining the vessel for a stabilized ternary complex including the polymerase and the nucleotide cognate of the second base type bound at the base position of the primed-template nucleic acid; (f) identifying the type of nucleotide at the base position of the primed-template nucleic acid; (g) delivering a nucleotide cognate of a third base type to the vessel, whereby the vessel retains the primed-template nucleic acid and the polymerase from step (b); (h) examining the vessel for a stabilized ternary complex having the polymerase and the nucleotide cognate of the third base type bound at the base position of the primed-template nucleic acid; (i) delivering a nucleotide cognate of a fourth base type to the vessel, whereby the vessel retains the primed-template nucleic acid and the polymerase from step (b); and (j) examining the vessel for a stabilized ternary complex including the polymerase and the nucleotide cognate of the fourth base type bound at the base position of the primed-template nucleic acid. Optionally, the method can further include steps of (k) adding a nucleotide to the primer of the primed-template nucleic acid, whereby the vessel includes an extended primed-template nucleic acid; (l) delivering a second polymerase and a nucleotide cognate of the first base type to the vessel; and (m) repeating steps (b) through (j) using the extended primed-template instead of the primed-template nucleic acid and using the second polymerase instead of the polymerase.

Also disclosed herein includes a composition which comprises a plurality of polymerase-nucleic acid complexes in contact with an aqueous solution, where each of the plurality of polymerase-nucleic acid complex comprises a polymerase and a primed-template nucleic acid, and where the aqueous solution comprises a polyol, alcohol, aliphatic diol, sulfone, sulfoxide, or a combination thereof. The aqueous solution can further comprise Lithium, Betaine, or both.

### BRIEF DESCRIPTION OF THE DRAWINGS

FIG. 1 shows a flow chart for an examination subroutine of a sequencing method.

FIG. 2 is a plot comparing crosstalk metrics measured for NSB containing isopropanol (SOP) compared to the crosstalk metric measured for NSB containing 1,6 hexanediol.

FIG. 3 is a plot comparing crosstalk metrics measured for NSB containing isopropanol (SOP) compared to the crosstalk metric measured for NSB containing 1,7 heptanediol.

FIG. 4 is a plot comparing crosstalk metrics measured for NSB containing isopropanol (SOP) compared to the crosstalk metric measured for NSB containing 1,5 pentanediol.

FIG. 5 is a plot comparing crosstalk metrics measured for NSB containing isopropanol (SOP) compared to the crosstalk metric measured for NSB containing DMSO.

FIG. 6 is a plot comparing crosstalk metrics measured for NSB containing isopropanol (SOP) compared to the crosstalk metric measured for NSB containing ethyl methyl sulfone.

FIG. 7 is a plot comparing crosstalk metrics measured for NSB containing isopropanol (SOP) compared to the crosstalk metric measured for NSB containing sulfolane.

FIG. 8 is a plot comparing crosstalk metrics measured for NSB containing isopropanol (SOP) compared to the crosstalk metric measured for NSB containing PEG.

FIG. 9 is a plot comparing crosstalk metrics measured for NSB containing isopropanol (SOP) compared to the crosstalk metric measured for NSB containing PVA.

FIG. 10A shows a first image acquired for NSB fluid foam having 28% 1,6 hexanediol; FIG. 10B shows a second image acquired 1 minute after the first image for NSB fluid foam having 28% 1,6 hexanediol; FIG. 10C shows a first image acquired for NSB fluid foam having 20% isopropanol (SOP); and FIG. 10D shows a second image acquired 1 minute after the first image for NSB fluid foam having 20% isopropanol (SOP).

### DETAILED DESCRIPTION

The present disclosure provides compositions and methods that can be used to modify molecular complexes, for example, by dissociating interactions between two or more of a polymerase, nucleic acid and nucleotide that participate in a ternary complex. The compositions and methods can be used for a variety of purposes including, for example, detecting the complexes, identifying characteristics of the complexes such as the identity of one or more bases in a nucleic acid that participates in the complex, producing complexes for use such as therapeutic use or diagnostic use, modifying complexes to remove a component from the complex or removing complexes entirely.

In some embodiments, the methods and compositions set forth herein can be used for identifying a nucleotide in a primed-template nucleic acid. The nucleotide can be identified based on formation of a ternary complex that includes the primed-template nucleic acid, a polymerase that binds to the template at the 3′ end of the primer and a cognate nucleotide that binds to the polymerase to pair with a nucleotide in the template that is adjacent to the 3′ end of the primer. A variety of different nucleotide types can be evaluated for the ability to form a ternary complex. The type of nucleotide that is observed to participate in formation of a ternary complex can be identified as the cognate nucleotide for the template position that is being queried. Based on this observation and the known rules for pairing nucleotides (i.e., adenine pairs with thymine or uracil, and cytosine pairs with guanine), the nucleotide type at the template position can be inferred.

A useful method for characterizing the primed-template nucleic acid is to deliver a polymerase and a first type of nucleotide to an immobilized nucleic acid, examine the solid support for recruitment of the ternary complex components to the immobilized nucleic acid, remove the polymerase and nucleotide from the solid support to which the nucleotide is immobilized, and then repeat the cycle for a different type of nucleotide. Although this method is useful for characterizing the nucleic acid, the delivery and removal of reagents from the solid support can be time consuming. Moreover, this replacement cycle consumes a relatively large amount of polymerase, which can be an expensive reagent to produce.

The present disclosure provides a method whereby different nucleotide types can be serially delivered and then removed from a vessel where a ternary complex is to be formed and examined. For example, each delivery can include only a single type of nucleotide or only nucleotide that is cognate for a single type of base expected to be in a nucleic acid. In another example, each delivery can include a set of at least 2, 3, 4 or more nucleotide types or a set of nucleotides that includes cognates for at least 2, 3, 4 or more base types expected to be in a nucleic acid. One or more nucleotides can be removed from a vessel under conditions that will dissociate a nucleotide from a ternary complex, thereby allowing the nucleotide to be separated from the primed-template nucleic acid without causing substantial removal of the polymerase. Another nucleotide can then be delivered to the primed-template nucleic acid. Delivery of more polymerase is not necessary if the polymerase is not substantially removed from the presence of the primed-template nucleic acid. This provides a savings of time and resources that would otherwise be spent preparing more polymerase.

Surprisingly, polyols, diols and sulfoxides have been shown to dissociate nucleotides from polymerase-nucleic acid complexes while retaining association between the polymerases and nucleic acids. As such, aqueous solutions that contain these compounds can be used to remove a nucleotide from a polymerase-nucleic acid complex or to replace one nucleotide for another in a polymerase-nucleic acid complex. More specifically, a ternary complex that includes a primed-template nucleic acid, polymerase and next correct nucleotide can be contacted with an aqueous solution that contains a polyol, diol, sulfone or sulfoxide in order to dissociate the nucleotide while the polymerase remains associated with the nucleic acid. For embodiments in which the polymerase-nucleic acid complex is immobilized on a surface in a vessel, the dissociated nucleotide can then be removed by removing the solution from the vessel. A new solution that contains the same or different type of nucleotide that was removed can then be added to the vessel under conditions for ternary complex formation.

The method can be performed in a multiplex fashion such that different nucleotides or sets of nucleotides are sequentially delivered and removed from a vessel or solid support having a plurality of polymerase-nucleic acid complexes. For example, a vessel that contains an array of polymerase-nucleic acid complexes can include a subset of complexes that form ternary complexes with the first type of nucleotide, the first nucleotide type can be dissociated from the ternary complexes and removed from the vessel using an aqueous solution containing a polyol, diol, sulfone or sulfoxide, and then a second nucleotide type can be delivered to the vessel such that a subset of the polymerase-nucleic acid complexes that have been retained in the vessel can form ternary complexes with the second type of nucleotide.

Although the embodiments described above are exemplified for delivery of a single type of nucleotide in each step, it will be understood that two or more nucleotide types can be delivered in one or more steps. The nucleotides can be distinguished, for example, using different labels attached to each type of nucleotide, respectively.

Terms used herein will be understood to take on their ordinary meaning in the relevant art unless specified otherwise. Several terms used herein, and their meanings are set forth below.

As used herein, the term “array” refers to a population of molecules attached to one or more solid supports such that the molecules at one feature can be distinguished from molecules at other features. An array can include different molecules that are each located at different addressable features on a solid support. Alternatively, an array can include separate solid supports each functioning as a feature that bears a different molecule, wherein the different molecules can be identified according to the locations of the solid supports on a surface to which the solid supports are attached, or according to the locations of the solid supports in a liquid such as a fluid stream. The molecules of the array can be, for example, nucleotides, nucleic acid primers, nucleic acid templates, primed-template nucleic acids, or nucleic acid enzymes such as polymerases, ligases, exonucleases or combinations thereof.

As used herein, the term “attached” refers to the state of two things being joined, fastened, adhered, connected or bound to each other. For example, a reaction component, such as a primed-template nucleic acid or a polymerase, can be attached to a solid phase component by a covalent or non-covalent bond. A covalent bond is characterized by the sharing of pairs of electrons between atoms. A non-covalent bond is a chemical bond that does not involve the sharing of pairs of electrons and can include, for example, hydrogen bonds, ionic bonds, van der Waals forces, hydrophilic interactions and hydrophobic interactions.

As used herein, the term “binary complex” refers to an intermolecular association between a polymerase and a nucleic acid (e.g., a primed-template nucleic acid), excluding any nucleotide molecule such as a next correct nucleotide for a primed-template nucleic acid.

As used herein, the term “blocking moiety,” when used in reference to a nucleotide, means a part of the nucleotide that inhibits or prevents the 3′ oxygen of the nucleotide from forming a covalent linkage to a next correct nucleotide during a nucleic acid polymerization reaction. The blocking moiety of a “reversibly terminated” nucleotide can be removed from the nucleotide analog, or otherwise modified, to allow the 3′-oxygen of the nucleotide to covalently link to a next correct nucleotide. Such a blocking moiety is referred to herein as a “reversible terminator moiety.” Exemplary reversible terminator moieties are set forth in U.S. Pat. Nos. 7,427,673; 7,414,116; 7,057,026; 7,544,794 or 8,034,923; or PCT publications WO 91/06678 published on May 16, 1991 or WO 07/123744 published on Nov. 1, 2007, each of which is incorporated herein by reference. A nucleotide that has a blocking moiety or reversible terminator moiety can be a subunit at the 3′ end of a nucleic acid, such as a primer, or the nucleotide can be a monomeric molecule that is not covalently attached to a nucleic acid. A particularly useful blocking moiety will be present at the 3′ end of a nucleic acid that participates in formation of a stabilized ternary complex.

As used herein, the term “catalytic metal ion” refers to a metal ion that facilitates phosphodiester bond formation between the 3′-oxygen of a nucleic acid (e.g., a primer) and the phosphate of an incoming nucleotide by a polymerase. A “divalent catalytic metal cation” is a catalytic metal ion having a valence of two. Catalytic metal ions can be present at concentrations that stabilize formation of a complex between a polymerase, nucleotide, and primed-template nucleic acid, referred to as non-catalytic concentrations of a metal ion insofar as phosphodiester bond formation does not occur. Catalytic concentrations of a metal ion refer to the amount of a metal ion sufficient for polymerases to catalyze the reaction between the 3′-oxygen of a nucleic acid (e.g., a primer) and the phosphate moiety of an incoming nucleotide.

The term “comprising” is intended herein to be open-ended, including not only the recited elements, but further encompassing any additional elements.

As used herein, the term “deblock” means to remove or modify a reversible terminator moiety of a nucleotide to render the nucleotide extendable. For example, the nucleotide can be present at the 3′ end of a primer such that deblocking renders the primer extendable. Exemplary deblocking reagents and methods are set forth in U.S. Pat. Nos. 7,427,673; 7,414,116; 7,057,026; 7,544,794 or 8,034,923; or PCT publications WO 91/06678 published on May 16, 1991 or WO 07/123744 published on Nov. 1, 2007, each of which is incorporated herein by reference.

As used herein, the term “diol” refers to a chemical compound containing two, and only two, hydroxyl moieties. The term “aliphatic diol” refers to a hydrocarbon containing two, and only two, hydroxyl moieties. The hydrocarbon can be saturated (i.e., having no carbon-carbon double bonds) or unsaturated (i.e., having at least one carbon-carbon double bond). Whether saturated or unsaturated, the hydrocarbon can be linear, branched or cyclic. The hydrocarbon can include at least 2, 3, 4, 5, 6, 7, 8 or more carbons. Alternatively or additionally, the hydrocarbon can include at most 8, 7, 6, 5, 4, 3 or 2 carbons. An aliphatic diol can also be referred to as a “glycol.” A diol can be a geminal diol, in which both hydroxyls are attached to the same carbon, non-geminal diol in which the hydroxyls are attached to different carbons, vicinal diols in which the hydroxyls are attached to adjacent carbons, non-vicinal diols in which the hydroxyls are not attached to adjacent carbons, or terminal diols in which the hydroxyls are attached at the ends of a linear hydrocarbon. The hydrocarbon of an aliphatic diol will not be aromatic. A diol having an aromatic hydrocarbon is referred to herein as an “aromatic diol.”

As used herein, the term “each,” when used in reference to a collection of items, is intended to identify an individual item in the collection but does not necessarily refer to every item in the collection. Exceptions can occur if explicit disclosure or context clearly dictates otherwise.

As used herein, the term “exogenous,” when used in reference to a moiety of a molecule, means a chemical moiety that is not present in a natural analog of the molecule. For example, an exogenous label of a nucleotide is a label that is not present on a naturally occurring nucleotide. Similarly, an exogenous label that is present on a polymerase is not found on the polymerase in its native milieu.

As used herein, the term “extendable,” when used in reference to a nucleotide, means that the nucleotide has an oxygen or hydroxyl moiety at the 3′ position, and is capable of forming a covalent linkage to a next correct nucleotide if and when incorporated into a nucleic acid. An extendable nucleotide can be a subunit at the 3′ position of a primer or it can be a monomeric molecule. A nucleotide that is extendable will lack blocking moieties such as reversible terminator moieties.

As used herein, the term “extended,” when used in reference to a primer or other nucleic acid, refers to the nucleic acid following incorporation of at least one nucleotide to the nucleic acid. The incorporation event can be, for example, polymerase catalyzed addition of one or more nucleotides to the 3′ end of the nucleic acid or ligase catalyzed addition of an oligonucleotide to the nucleic acid.

As used herein, the term “extension,” when used in reference to a nucleic acid, means a process of adding at least one nucleotide to the 3′ end or 5′ end of the nucleic acid. The term “polymerase extension,” when used in reference to a nucleic acid, refers to a polymerase catalyzed process of adding at least one nucleotide to the 3′ end of the nucleic acid. A nucleotide or oligonucleotide that is added to a nucleic acid by extension is said to be incorporated into the nucleic acid. Accordingly, the term “incorporating” can be used to refer to the process of j oining a nucleotide or oligonucleotide to the 3′ end or 5′ end of a nucleic acid by formation of a phosphodiester bond.

As used herein, the term “feature,” when used in reference to an array, means a location in an array where a particular molecule is present. A feature can contain only a single molecule or it can contain a population of several molecules of the same species (i.e., an ensemble of the molecules). Alternatively, a feature can include a population of molecules that are different species (e.g., a population of ternary complexes having different template sequences). Features of an array are typically discrete. The discrete features can be contiguous or they can have spaces between each other. An array useful herein can have, for example, features that are separated by less than 100 microns, 50 microns, 10 microns, 5 microns, 1 micron, or 0.5 micron. Alternatively or additionally, an array can have features that are separated by greater than 0.5 micron, 1 micron, 5 microns, 10 microns, 50 microns or 100 microns. The features can each have an area of less than 1 square millimeter, 500 square microns, 100 square microns, 25 square microns, 1 square micron or less.

As used herein, a “flow cell” is a vessel that includes one or more channels that have a detection zone. The detection zone can be coupled to a detector such that a reaction occurring in the vessel can be observed. For example, a flow cell can contain primed-template nucleic acid molecules tethered to a solid support, to which nucleotides and ancillary reagents are iteratively applied and washed away. The detection zone can include a transparent material that permits the sample to be imaged after a desired reaction occurs. For example, a flow cell can include a glass or plastic slide containing small fluidic channels through which polymerases, dNTPs and buffers can be pumped. The glass or plastic inside the channels can be decorated with one or more primed-template nucleic acid molecules to be sequenced. An external imaging system can be positioned to detect the molecules at a detection zone. Exemplary flow cells, methods for their manufacture and methods for their use are described in US Pat. App. Publ. Nos. 2010/0111768 A1 published on May 6, 2010 or 2012-0270305 A1 published on Oct. 5, 2012; or WO 05/065814 published on Jul. 21, 2005, each of which is incorporated by reference herein.

As used herein, the term “immobilized,” when used in reference to a molecule, refers to direct or indirect, covalent or non-covalent attachment of the molecule to a surface such as a surface of a solid support. In some embodiments, covalent attachment may be preferred, but generally all that is required is that the molecules (e.g., nucleic acids) remain immobilized or attached to the surface under the conditions in which surface retention is intended.

As used herein, the term “label” refers to a molecule, or moiety thereof, that provides a detectable characteristic. The detectable characteristic can be, for example, an optical signal such as absorbance of radiation, fluorescence emission, luminescence emission, fluorescence lifetime, luminescence lifetime, fluorescence polarization, luminescence polarization or the like; Rayleigh and/or Mie scattering; binding affinity for a ligand or receptor; magnetic properties; electrical properties; charge; mass; radioactivity or the like. Exemplary labels include, without limitation, a fluorophore, luminophore, chromophore, nanoparticle (e.g., gold, silver, carbon nanotubes), heavy atoms, radioactive isotope, mass label, charge label, spin label, receptor, ligand, or the like.

As used herein, the term “next correct nucleotide” refers to the nucleotide (or nucleotide type) that will bind and/or incorporate at the 3′ end of a primer to complement a base in a template strand to which the primer is hybridized. The base in the template strand is referred to as the “next base” and is immediately 5′ of the base in the template that is hybridized to the 3′ end of the primer. The next correct nucleotide can be referred to as the “cognate” of the next base and vice versa. Cognate nucleotides that interact with each other in a ternary complex or in a double stranded nucleic acid are said to “pair” with each other. In accordance with Watson-Crick pairing rules adenine (A) pairs with thymine (T) or uracil (U), and cytosine (C) pairs with guanine (G). A nucleotide having a base that is not complementary to the next template base is referred to as an “incorrect”, “mismatch” or “non-cognate” nucleotide for the next template base.

As used herein, the term “non-catalytic metal ion” refers to a metal ion that, when in the presence of a polymerase enzyme, does not facilitate phosphodiester bond formation needed for chemical incorporation of a nucleotide into a primer. A non-catalytic metal ion may interact with a polymerase, for example, via competitive binding compared to catalytic metal ions. Accordingly, a non-catalytic metal ion can act as an inhibitory metal ion. A “divalent non-catalytic metal ion” is a non-catalytic metal ion having a valence of two. Examples of divalent non-catalytic metal ions include, but are not limited to, Ca²⁺, Zn²⁺, Co²⁺, Ni²⁺, and Sr²⁺. The trivalent Eu³⁺ and Tb³⁺ ions are non-catalytic metal ions having a valence of three.

As used herein, the term “nucleotide” can be used to refer to a native nucleotide or analog thereof. Examples include, but are not limited to, nucleotide triphosphates (NTPs) such as ribonucleotide triphosphates (rNTPs), deoxyribonucleotide triphosphates (dNTPs), or non-natural analogs thereof such as dideoxyribonucleotide triphosphates (ddNTPs) or reversibly terminated nucleotide triphosphates (rtNTPs).

As used herein, the term “polymerase” can be used to refer to a nucleic acid synthesizing enzyme, including but not limited to, DNA polymerase, RNA polymerase, reverse transcriptase, primase and transferase. Typically, the polymerase has one or more active sites at which nucleotide binding and/or catalysis of nucleotide polymerization may occur. The polymerase may catalyze the polymerization of nucleotides to the 3′ end of the first strand of the double stranded nucleic acid molecule. For example, a polymerase catalyzes the addition of a next correct nucleotide to the 3′ oxygen moiety of the first strand of the double stranded nucleic acid molecule via a phosphodiester bond, thereby covalently incorporating the nucleotide to the first strand of the double stranded nucleic acid molecule. Optionally, a polymerase need not be capable of nucleotide incorporation under one or more conditions used in a method set forth herein. For example, a mutant polymerase may be capable of forming a ternary complex but incapable of catalyzing nucleotide incorporation.

As used herein, the term “polymerase-nucleic acid complex” refers to an intermolecular association between a polymerase and a nucleic acid. Exemplary polymerase-nucleic acid complexes include, but are not limited to a binary complex, ternary complex or stabilized ternary complex.

As used herein, the term “polyol” refers to an organic compound containing multiple hydroxyl moieties. Exemplary polyols include diols (i.e., having 2, and only 2, hydroxyl moieties), triols (i.e., having 3, and only 3, hydroxyl moieties), and tetrols (i.e., having 4, and only 4, hydroxyl moieties).

As used herein, the term “primed-template nucleic acid” or “primed-template” refers to a nucleic acid having a double stranded region such that one of the strands functions as a primer and the other strand functions as a template. The two strands can be parts of a contiguous nucleic acid molecule (e.g., a hairpin structure) or the two strands can be separable molecules that are not covalently attached to each other.

As used herein, the term “primer” refers to a nucleic acid having a sequence that binds to a nucleic acid at or near a template sequence. Generally, the primer binds in a configuration that allows replication of the template, for example, via polymerase extension of the primer. The primer can be a first portion of a nucleic acid molecule that binds to a second portion of the nucleic acid molecule, the first portion being a primer sequence and the second portion being a primer binding sequence (e.g., a hairpin primer). Alternatively, the primer can be a first nucleic acid molecule that binds to a second nucleic acid molecule having the template sequence. A primer can consist of DNA, RNA or analogs thereof. A primer can have an extendible 3′ end or a 3′ end that is blocked from primer extension.

As used herein, the term “signal” refers to energy or coded information that can be selectively observed compared to other energy or information such as background energy or noise. A signal can have a desired or predefined characteristic. For example, an optical signal can be characterized or observed by one or more of intensity, wavelength, energy, frequency, power, luminance or the like. Other signals can be characterized according to characteristics such as voltage, current, electric field strength, magnetic field strength, frequency, power, temperature, etc. An optical signal can be detected at a particular intensity, wavelength, or color; an electrical signal can be detected at a particular frequency, power or field strength; or other signals can be detected based on characteristics known in the art pertaining to spectroscopy and analytical detection. Absence of signal is understood to be a signal level of zero or a signal level that is not meaningfully distinguished from background noise.

As used herein, the term “solid support” refers to a rigid substrate that is substantially insoluble in liquids that it contacts. The substrate can be non-porous or porous. The substrate can optionally be capable of taking up a liquid (e.g., due to porosity) but will typically be sufficiently rigid that the substrate does not swell substantially when taking up the liquid and does not contract substantially when the liquid is removed by drying. A nonporous solid support is generally impermeable to liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefins, polyimides etc.), nylon, ceramics, resins, Zeonor, silica or silica-based materials including silicon and modified silicon, carbon, metals, inorganic glasses, optical fiber bundles, and polymers. Any of a variety of liquids, including but not limited to those set forth herein, can be contacted with a solid support.

As used herein, the term “subset” means a collection of one or more things, all of which are contained in a larger collection of things. The larger collection of things can be referred to as a “set”. A subset can include at least 1, 2, 10, 100, 1×10³, 1×10⁶, 1×10⁹ or more things. A subset can include at least 10%, 25%, 50%, 75%, 90%, 99% or more of the things that are in the set. Alternatively or additionally, a subset can include at most 99%, 90%, 80%, 75%, 70%, 65%, 60%, 55%, 50%, 40%, 30%, 25%, 10%, or fewer of the things that are in the set. The things can be, for example, nucleic acids such as primers, templates or primer-template nucleic acids; ternary complexes such as stabilized ternary complexes, polymerases, nucleotides or other compositions set forth herein.

As used herein, the term “sulfone” refers to a chemical compound having structure R—S(═O)₂—R′, where R and R′ are organic moieties. The organic moieties can optionally be hydrocarbons such as aliphatic chains or aromatic chains. The aliphatic chains or aromatic chains can be linear, branched or cyclical. The organic moieties can contain heteroatoms such as nitrogen, oxygen or the like. R and R′ can be the same type of moiety, different types of moieties, or attachments to a single moiety such as a ring structure.

As used herein, the term “sulfoxide” refers to a chemical compound having structure R—S(═O)—R′, where R and R′ are organic moieties. The organic moieties can optionally be hydrocarbons such as aliphatic chains or aromatic chains. The aliphatic chains or aromatic chains can be linear, branched or cyclical. The organic moieties can contain heteroatoms such as nitrogen, oxygen or the like. R and R′ can be the same type of moiety, different types of moieties, or attachments to a single moiety such as a ring structure.

As used herein, the term “ternary complex” refers to an intermolecular association between a polymerase, a double stranded nucleic acid and a nucleotide. Typically, the polymerase facilitates interaction between a next correct nucleotide and a template strand of the primed nucleic acid. A next correct nucleotide can interact with the template strand via Watson-Crick hydrogen bonding. The term “stabilized ternary complex” means a ternary complex having promoted or prolonged existence or a ternary complex for which disruption has been inhibited. Generally, stabilization of the ternary complex prevents covalent incorporation of the nucleotide component of the ternary complex into the primed nucleic acid component of the ternary complex.

As used herein, the term “type” is used to identify molecules that share the same chemical structure. For example, a mixture of nucleotides can include several dCTP molecules. The dCTP molecules will be understood to be the same type of nucleotide as each other, but a different type of nucleotide compared to dATP, dGTP, dTTP etc. Similarly, individual DNA molecules that have the same sequence of nucleotides are the same type, whereas DNA molecules with different sequences are different types. The term “type” can also identify moieties that share the same chemical structure. For example, the cytosine bases in a template nucleic acid will be understood to be the same type of base as each other independent of their position in the template sequence.

As used herein, a “vessel” is a container that functions to isolate one chemical process (e.g., a binding event; an incorporation reaction; etc.) from another, or to provide a space in which a chemical process can take place. Examples of vessels useful in connection with the disclosed technique include, but are not limited to, flow cells, wells of a multi-well plate; microscope slides; tubes (e.g., capillary tubes); droplets, vesicles, test tubes, trays, centrifuge tubes, features in an array, tubing, channels in a substrate etc. As used herein, a “manufactured vessel” is a container that is human-made or human-modified and that functions to isolate one chemical process (e.g., a binding event; an incorporation reaction; etc.) from another, or to provide a space in which a chemical process can take place.

The embodiments set forth below and recited in the claims can be understood in view of the above definitions.

The present disclosure provides a composition, including a polymerase-nucleic acid complex, wherein the nucleic acid includes a primed-template nucleic acid in contact with an aqueous solution, wherein the aqueous solution includes a polyol, diol, sulfone or sulfoxide. The polymerase-nucleic acid complex can comprise a polymerase and a primed-template nucleic acid, and optionally a nucleotide in a ternary complex.

A polymerase-nucleic acid complex of a composition or method set forth herein can be in any of a variety of states. For example, the complex can be a binary complex that includes a polymerase bound to a nucleic acid. The nucleic acid can be a primed-template nucleic acid and optionally the polymerase can be bound at or near the 3′ end of the primer component of the primed-template. Another example of a polymerase-nucleic acid complex is a ternary complex which includes a polymerase bound to a nucleic acid and further includes a nucleotide. The nucleic acid is typically a primed-template nucleic acid and the polymerase is typically bound at or near the 3′ end of the primer such that the nucleotide base pairs with the next template base of the template nucleic acid. In this configuration, the nucleotide is the next correct nucleotide, being a cognate for the next template base.

In some embodiments of the compositions and methods set forth herein, the polymerase-nucleic acid complex is a ternary complex (for example, a stabilized ternary complex). The primer strand of a primed-template nucleic acid molecule that is present in a stabilized ternary complex is chemically unchanged by a polymerase that is present during one or more steps of a method set forth herein. For example, the primer in a stabilized ternary complex need not be extended by formation of a new phosphodiester bond, nor shortened by nucleolytic degradation during one or more steps of a method set forth herein, for example, during a step for forming a stabilized ternary complex, for dissociating a stabilized ternary complex or for detecting a stabilized ternary complex.

A ternary complex can be stabilized by any of a variety of means. While a ternary complex can form between a polymerase, primed-template nucleic acid and next correct nucleotide in the absence of certain catalytic metal ions (e.g., Mn²⁺ or Mg²⁺), chemical addition of the nucleotide is inhibited in the absence of catalytic metal ions. Low or deficient levels of catalytic metal ions cause non-covalent sequestration of the next correct nucleotide in a stabilized ternary complex.

Optionally, a stabilized ternary complex can be formed when the primer of a primed-template nucleic acid includes a blocking moiety (e.g., a reversible terminator moiety) that precludes enzymatic incorporation of an incoming nucleotide into the primer. The interaction can take place in the presence of stabilizers, whereby the polymerase-nucleic acid interaction is stabilized in the presence of the next correct nucleotide. The primer of the primed-template nucleic acid optionally can be either an extendable primer, or a primer blocked from extension at its 3′-end (e.g., blocking can be achieved by the presence of a reversible terminator moiety on the 3′-end of the primer). The primed-template nucleic acid, the polymerase and the cognate nucleotide are capable of forming a stabilized ternary complex when the base of the next correct nucleotide is complementary to the next base of the primed-template nucleic acid.

As set forth above, conditions that favor or stabilize a ternary complex can be provided by the presence of a blocking moiety that precludes enzymatic incorporation of an incoming nucleotide into the primer (e.g., a reversible terminator moiety on the 3′ nucleotide of the primer) or by the absence of a catalytic metal ion. Other useful conditions include the presence of a ternary complex stabilizing agent such as a non-catalytic ion (e.g., a divalent or trivalent non-catalytic metal ion) that inhibits nucleotide incorporation. Non-catalytic metal ions include, but are not limited to, calcium, strontium, scandium, titanium, vanadium, chromium, iron, cobalt, nickel, copper, zinc, gallium, germanium, arsenic, selenium, rhodium, europium, and terbium ions. As a further option, a polymerase engineered to prevent catalytic activity can be used. Examples of polymerases so engineered are set forth in U.S. Pat. Nos. 10,584,379 and 10,597,643, each of which is incorporated herein by reference.

It will be understood that options set forth herein for stabilizing a ternary complex need not be mutually exclusive and instead can be used in various combinations. For example, a ternary complex can be stabilized by one or a combination of means including, but not limited to, crosslinking of the polymerase domains, crosslinking of the polymerase to the nucleic acid, polymerase mutations that stabilize the ternary complex, an allosteric inhibitor of polymerase activity, an uncompetitive inhibitor of polymerase activity, a competitive inhibitor of polymerase activity, a non-competitive inhibitor of polymerase activity, absence of catalytic metal ions, presence of a blocking moiety on the primer, and other means set forth herein. In some embodiments of the methods or compositions set forth herein, the polymerase is not covalently attached to other components of the ternary complex. Moreover, a polymerase can be, but need not be, covalently attached to a solid support, such as a substrate used for an array of nucleic acids. For example, the polymerase can be free to diffuse in solution but for its non-covalent affinity for components of the ternary complex that are attached to a solid support.

A ternary complex of the present disclosure, whether stabilized or not, may optionally include one or more exogenous label(s). The label can be attached to a component of the ternary complex (e.g., attached to the polymerase, template nucleic acid, primer and/or cognate nucleotide) prior to formation of the ternary complex. Exemplary attachments include covalent attachments or non-covalent attachments such as those set forth herein, in references cited herein or known in the art. In some embodiments, a labeled component is delivered in solution to a solid support that is attached to an unlabeled component, whereby the label is recruited to the solid support by virtue of forming a ternary complex (e.g., a stabilized ternary complex). As such, the support-attached component can be detected or identified based on observation of the recruited label. Whether used in solution phase or on a solid support, exogenous labels can be useful for detecting a ternary complex or an individual component thereof, for example, during a step of examining a stabilized ternary complex in a method set forth herein. An exogenous label can remain attached to a component after the component dissociates from a stabilized ternary complex. Exemplary labels, methods for attaching labels and methods for using labeled components are set forth in U.S. Pat. App. Pub. Nos. 2017/0022553 A1 published on Jan. 26, 2017; 2018/0044727 A1 published on Feb. 15, 2018; 2018/0187245 A1 published on Jul. 5, 2018; and 2018/0208983 A1 published on Jul. 26, 2018, each of which is incorporated herein by reference.

Any of a variety of polymerases can be used in a method or composition set forth herein, for example, to form a polymerase-nucleic acid complex or to carry out primer extension. Polymerases that may be used include naturally occurring polymerases and modified variations thereof, including, but not limited to, mutants, recombinants, fusions, genetic modifications, chemical modifications, synthetics, and analogs. Naturally occurring polymerases and modified variations thereof are not limited to polymerases that have the ability to catalyze a polymerization reaction. Optionally, the naturally occurring and/or modified variations thereof have the ability to catalyze a polymerization reaction in at least one condition that is not used during formation or examination of a stabilized ternary complex. Optionally, the naturally-occurring and/or modified variations that participate in polymerase-nucleic acid complexes have modified properties, for example, enhanced binding affinity to nucleic acids, reduced binding affinity to nucleic acids, enhanced binding affinity to nucleotides, reduced binding affinity to nucleotides, enhanced specificity for next correct nucleotides, reduced specificity for next correct nucleotides, reduced catalysis rates, catalytic inactivity etc. Mutant polymerases include, for example, polymerases wherein one or more amino acids are replaced with other amino acids, or insertions or deletions of one or more amino acids. Exemplary polymerase mutants that can be used to form a stabilized ternary complex include, for example, those set forth in US Pat. App. Pub. No. 2020/0087637 A1 published on Mar. 19, 2020 and U.S. Pat. Nos. 10,584,379 and 10,597,643, each of which is incorporated herein by reference.

Modified polymerases include polymerases that contain an exogenous label moiety (e.g., an exogenous fluorophore), which can be used to detect the polymerase. Optionally, the label moiety can be attached after the polymerase has been at least partially purified using protein isolation techniques. For example, the exogenous label moiety can be covalently linked to the polymerase using a free sulfhydryl or a free amine moiety of the polymerase. This can involve covalent linkage to the polymerase through the side chain of a cysteine residue, or through the free amino moiety of the N-terminus. An exogenous label moiety can also be attached to a polymerase via protein fusion. Exemplary label moieties that can be attached via protein fusion include, for example, green fluorescent protein (GFP), phycobiliproteins (e.g., phycocyanin and phycoerythrin) or wavelength-shifted variants of GFP or phycobiliproteins.

Alternatively, a polymerase that participates in a polymerase-nucleic acid complex, or that is used to extend a primer, need not be attached to an exogenous label. For example, the polymerase need not be covalently attached to an exogenous label. Instead, the polymerase can lack any label until it, optionally, associates with a labeled nucleotide and/or labeled nucleic acid (e.g., labeled primer and/or labeled template).

Different activities of polymerases can be exploited in a method set forth herein. A polymerase can be useful, for example, in a primer extension step, examination step or combination thereof. The different activities can follow from differences in the structure (e.g., via natural activities, mutations or chemical modifications). Nevertheless, polymerase can be obtained from a variety of known sources and applied in accordance with the teachings set forth herein and recognized activities of polymerases. Useful DNA polymerases include, but are not limited to, bacterial DNA polymerases, eukaryotic DNA polymerases, archaeal DNA polymerases, viral DNA polymerases and phage DNA polymerases. Bacterial DNA polymerases include E. coli DNA polymerases I, II and III, IV and V, the Klenow fragment of E. coli DNA polymerase, Clostridium stercorarium (Cst) DNA polymerase, Clostridium thermocellum (Cth) DNA polymerase and Sulfolobus solfataricus (Sso) DNA polymerase. Eukaryotic DNA polymerases include DNA polymerases α, β, γ, δ, €, η, ζ, λ, σ, μ, and k, as well as the Revl polymerase (terminal deoxycytidyl transferase) and terminal deoxynucleotidyl transferase (TdT). Viral DNA polymerases include T4 DNA polymerase, phi-29 DNA polymerase, GA-1, phi-29-like DNA polymerases, PZA DNA polymerase, phi-15 DNA polymerase, Cpl DNA polymerase, Cpl DNA polymerase, T7 DNA polymerase, and T4 polymerase. Other useful DNA polymerases include thermostable and/or thermophilic DNA polymerases such as Thermus aquaticus (Taq) DNA polymerase, Thermus filiformis (Tfi) DNA polymerase, Thermococcus zilligi (Tzi) DNA polymerase, Thermus thermophilus (Tth) DNA polymerase, Thermus flavusu (Tfl) DNA polymerase, Pyrococcus woesei (Pwo) DNA polymerase, Pyrococcus furiosus (Pfu) DNA polymerase and Turbo Pfu DNA polymerase, Thermococcus litoralis (Tli) DNA polymerase, Pyrococcus sp. GB-D polymerase, Thermotoga maritima (Tma) DNA polymerase, Bacillus stearothermophilus (Bst) DNA polymerase, Pyrococcus Kodakaraensis (KOD) DNA polymerase, Pfx DNA polymerase, Thermococcus sp. JDF-3 (JDF-3) DNA polymerase, Thermococcus gorgonarius (Tgo) DNA polymerase, Thermococcus acidophilium DNA polymerase; Sulfolobus acidocaldarius DNA polymerase; Thermococcus sp. go N-7 DNA polymerase; Pyrodictium occultum DNA polymerase; Methanococcus voltae DNA polymerase; Methanococcus thermoautotrophicum DNA polymerase; Methanococcus jannaschii DNA polymerase; Desulfurococcus strain TOK DNA polymerase (D. Tok Pol); Pyrococcus abyssi DNA polymerase; Pyrococcus horikoshii DNA polymerase; Pyrococcus islandicum DNA polymerase; Thermococcus fumicolans DNA polymerase; Aeropyrum pernix DNA polymerase; and the heterodimeric DNA polymerase DP1/DP2. Engineered and modified polymerases also are useful in connection with the disclosed techniques. For example, modified versions of the extremely thermophilic marine archaea Thermococcus species 9° N (e.g., Therminator DNA polymerase from New England BioLabs Inc.; Ipswich, Mass.) can be used.

Useful RNA polymerases include, but are not limited to, viral RNA polymerases such as T7 RNA polymerase, T3 polymerase, SP6 polymerase, and Kll polymerase; Eukaryotic RNA polymerases such as RNA polymerase I, RNA polymerase II, RNA polymerase III, RNA polymerase IV, and RNA polymerase V; and Archaea RNA polymerase.

Another useful type of polymerase is a reverse transcriptase. Exemplary reverse transcriptases include, but are not limited to, HIV-1 reverse transcriptase from human immunodeficiency virus type 1 (PDB 1HMV), HIV-2 reverse transcriptase from human immunodeficiency virus type 2, M-MLV reverse transcriptase from the Moloney murine leukemia virus, AMV reverse transcriptase from the avian myeloblastosis virus, and Telomerase reverse transcriptase that maintains the telomeres of eukaryotic chromosomes.

A polymerase having an intrinsic 3′-5′ proofreading exonuclease activity can be useful for some embodiments. Polymerases that substantially lack 3′-5′ proofreading exonuclease activity are also useful in some embodiments, for example, in most genotyping and sequencing embodiments. Absence of exonuclease activity can be a wild type characteristic or a characteristic imparted by a variant or engineered polymerase structure. For example, exo minus Klenow fragment is a mutated version of Klenow fragment that lacks 3′-5′ proofreading exonuclease activity. Klenow fragment and its exo minus variant can be useful in a method or composition set forth herein.

Nucleic acids that are used in a method or composition set forth herein can be DNA such as genomic DNA, synthetic DNA, amplified DNA, complementary DNA (cDNA) or the like. RNA can also be used such as mRNA, ribosomal RNA, tRNA or the like. Nucleic acid analogs can also be used as templates herein. Thus, template nucleic acids used herein can be derived from a biological source, synthetic source or amplification product. Primers used herein can be DNA, RNA or analogs thereof.

Particularly useful nucleic acid templates are genome fragments that each include a sequence identical to a portion of a genome. A population of genome fragments can cover all or part of the sequence of a particular genome. For example, a population of genome fragments can include sequences for at least 5%, 10%, 20%, 30%, 40%, 50%, 60%, 70%, 75%, 80%, 85%, 90%, 95% or 99% of a genome. A genome fragment can have, for example, a sequence that is substantially identical to at least about 25, 50, 70, 100, 200, 300, 400, 500, 600, 700, 800, 900 or 1000 or more contiguous nucleotides of a genome. Alternatively or additionally, a genome fragment can have a sequence that is substantially identical to no more than 1×10⁵, 1×10⁴, 1×10³, 800, 600, 400, 200, 100, 75, 50 or 25 contiguous nucleotides of a genome. A genome fragment can be DNA, RNA, or an analog thereof.

Exemplary organisms from which nucleic acids can be derived include, for example, a mammal such as a rodent, mouse, rat, rabbit, guinea pig, ungulate, horse, sheep, pig, goat, cow, cat, dog, primate, human or non-human primate; a plant such as Arabidopsis thaliana, corn, sorghum, oat, wheat, rice, canola, or soybean; an algae such as Chlamydomonas reinhardtii; a nematode such as Caenorhabditis elegans; an insect such as Drosophila melanogaster, mosquito, fruit fly, honey bee or spider; a fish such as zebrafish; a reptile; an amphibian such as a frog or Xenopus laevis; a Dictyostelium discoideum; a fungi such as Pneumocystis carinii, Takifugu rubripes, yeast, Saccharamoyces cerevisiae or Schizosaccharomyces pombe; or a Plasmodium falciparum. Nucleic acids can also be derived from a prokaryote such as a bacterium, Escherichia coli, Staphylococci or Mycoplasma pneumoniae; an archae; a virus such as Hepatitis C virus, influenza virus, corona virus or human immunodeficiency virus; or a viroid. Nucleic acids can be derived from a homogeneous culture or population of the above organisms or alternatively from a collection of several different organisms, for example, in a community or ecosystem. Nucleic acids can be isolated using methods known in the art including, for example, those described in Sambrook et al., Molecular Cloning: A Laboratory Manual, 3rd edition, Cold Spring Harbor Laboratory, New York (2001) or in Ausubel et al., Current Protocols in Molecular Biology, John Wiley and Sons, Baltimore, Md. (1998), each of which is incorporated herein by reference.

A nucleic acid can be obtained from a preparative method such as genome isolation, genome fragmentation, gene cloning and/or amplification. The nucleic acid can be obtained from an amplification technique such as polymerase chain reaction (PCR), rolling circle amplification (RCA), multiple displacement amplification (MDA) or the like. Exemplary methods for isolating, amplifying and fragmenting nucleic acids to produce templates for molecular analysis are set forth in U.S. Pat. Nos. 6,355,431 or 9,045,796, each of which is incorporated herein by reference. Amplification can also be carried out using a method set forth in Sambrook et al., Molecular Cloning: A Laboratory Manual, 3rd edition, Cold Spring Harbor Laboratory, New York (2001) or in Ausubel et al., Current Protocols in Molecular Biology, John Wiley and Sons, Baltimore, Md. (1998), each of which is incorporated herein by reference.

A composition or method set forth herein can include a native nucleotide, nucleotide analog or modified nucleotide as desired to suit a particular application or some embodiments of the methods. Optionally, a nucleotide analog has a nitrogenous base, five-carbon sugar, and phosphate moiety, wherein any moiety of the nucleotide may be modified, removed and/or replaced as compared to a native nucleotide. Nucleotide analogs may be non-incorporable nucleotides (i.e., nucleotides that are incapable of reacting with the 3′ oxygen of a primer to form a covalent linkage). Such nucleotides that are incapable of incorporation include, for example, monophosphate and diphosphate nucleotides. In another example, the nucleotide may contain modification(s) at the 5′ position (e.g., at the triphosphate moiety) that make the nucleotide non-incorporable. Examples of non-incorporable nucleotides may be found in U.S. Pat. Nos. 7,482,120 or 8,632,975, each of which is incorporated by reference herein. In some embodiments, non-incorporable nucleotides may be subsequently modified to become incorporable. Non-incorporable nucleotide analogs include, but are not limited to, alpha-phosphate modified nucleotides, alpha-beta nucleotide analogs, beta-phosphate modified nucleotides, beta-gamma nucleotide analogs, gamma-phosphate modified nucleotides, nucleotides having a 5′ phosphorothioate moiety, or caged nucleotides. Further examples of nucleotide analogs are described in U.S. Pat. No. 8,071,755, which is incorporated by reference herein.

Nucleotide analogs can include terminators that reversibly prevent subsequent nucleotide incorporation at the 3′-end of the primer after the analog has been incorporated into the primer. For example, U.S. Pat. Nos. 7,544,794 and 8,034,923 (the disclosures of these patents are incorporated herein by reference) describe reversible terminators in which the 3′-OH moiety is replaced by a 3′-ONH₂ moiety. Another type of reversible terminator is linked to the nitrogenous base of a nucleotide as set forth, for example, in U.S. Pat. No. 8,808,989 (the disclosure of which is incorporated herein by reference). Other reversible terminators that similarly can be used in connection with the methods described herein include an azido methyl moiety or others described in references cited elsewhere herein or in U.S. Pat. Nos. 7,956,171; 8,071,755, and 9,399,798, each of which is incorporated herein by reference. In certain embodiments, a reversible terminator moiety can be modified or removed from a primer, in a process known as “deblocking,” allowing for subsequent nucleotide incorporation. Compositions and methods for deblocking are set forth in references cited herein in the context of reversible terminators.

Alternatively, nucleotide analogs irreversibly prevent nucleotide incorporation at the 3′-end of the primer to which they have been incorporated. Irreversible nucleotide analogs include 2′, 3′-dideoxynucleotides (ddNTPs such as ddGTP, ddATP, ddTTP, ddCTP). Dideoxynucleotides lack the 3′-OH moiety of dNTPs that would otherwise participate in polymerase-mediated primer extension. Thus, the 3′ position has a hydrogen moiety instead of the native hydroxyl moiety. Irreversibly terminated nucleotides can be particularly useful for genotyping applications or other applications where primer extension or sequential detection along a template nucleic acid is not desired.

In some embodiments, a nucleotide that is used in a method or composition set forth herein can include an exogenous label such as a luminophore. Optionally, an exogenously labeled nucleotide can include a reversible or irreversible terminator moiety, an exogenously labeled nucleotide can be non-incorporable, an exogenously labeled nucleotide can lack blocking moieties, an exogenously labeled nucleotide can be incorporable or an exogenously labeled nucleotide can be both incorporable and non-terminated. Exogenously labeled nucleotides can be particularly useful when used to form a stabilized ternary complex with a non-labeled polymerase. For example, the label can produce luminescence that is detected in a method set forth herein. Alternatively, an exogenous label on a nucleotide can provide one partner in a fluorescence resonance energy transfer (FRET) pair and an exogenous label on a polymerase can provide the second partner of the pair. As such, FRET detection can be used to identify a stabilized ternary complex that includes both partners.

Alternatively, a nucleotide that participates in forming a ternary complex can lack exogenous labels (i.e., the nucleotide can be “non-labeled”). Optionally, a non-labeled nucleotide can include a reversible or irreversible terminator moiety, a non-labeled nucleotide can be non-incorporable, a non-labeled nucleotide can lack terminator moieties, a non-labeled nucleotide can be incorporable, or a non-labeled nucleotide can be both incorporable and non-terminated. Non-labeled nucleotides can be useful when a label on a polymerase is used to detect a stabilized ternary complex. Non-labeled nucleotides can also be useful in an extension step of a method set forth herein. It will be understood that absence of a moiety or function for a nucleotide refers to the nucleotide having substantially no such function or moiety. It will also be understood that one or more of the functions or moieties set forth herein for a nucleotide, or analog thereof, or otherwise known in the art for a nucleotide, or analog thereof, can be specifically omitted in a method or composition set forth herein.

Optionally, a composition or method set forth herein includes one or more different types of nucleotides (e.g., a native nucleotide or synthetic nucleotide analog). For example, at least 1, 2, 3, 4 or more nucleotide types can be present. Alternatively or additionally, at most 4, 3, 2, or 1 nucleotide types can be present. Similarly, one or more nucleotide types that are present can be cognates for at least 1, 2, 3 or 4 base types in a template nucleic acid. Alternatively or additionally, one or more nucleotide types that are present can be cognates for at most 4, 3, 2, or 1 base types in a template nucleic acid.

In some embodiments, a nucleotide will not have modifications that prevent participation in a ternary complex (e.g., a stabilized ternary complex). The nucleotide may be bound permanently or transiently to a polymerase. Optionally, a nucleotide analog is fused to a polymerase, for example, via a covalent linker. Optionally, a plurality of nucleotide analogs is fused to a plurality of polymerases, wherein each nucleotide analog is fused to a different polymerase. Optionally, a nucleotide that is present in a stabilized ternary complex is not the means by which the ternary complex is stabilized. Accordingly, any of a variety of other ternary complex stabilization methods may be combined in a reaction utilizing a nucleotide analog.

In some embodiments of the methods or compositions set forth herein, a polymerase-nucleic acid complex or components of the complex such as a nucleotide, polymerase or nucleic acid can include an exogenous label. An exogenous label can be attached to or associated with any of a variety of molecules, reagents, solid supports, vessels or other items that are to be detected.

Examples of useful exogenous labels include, but are not limited to, radiolabel moieties, luminophore moieties, fluorophore moieties, quantum dot moieties, chromophore moieties, enzyme moieties, electromagnetic spin labeled moieties, nanoparticle light scattering moieties, and any of a variety of other signal generating moieties known in the art. Suitable enzyme moieties include, for example, horseradish peroxidase, alkaline phosphatase, beta-galactosidase, or acetylcholinesterase. Exemplary fluorophore moieties include, but are not limited to umbelliferone, fluorescein, isothiocyanate, rhodamine, tetramethyl rhodamine, eosin, green fluorescent protein and wavelength shifted variants thereof, erythrosin, coumarin, methyl coumarin, pyrene, malachite green, stilbene, Lucifer Yellow™, Cascade Blue™, Texas Red™, DyLight® dyes, CF® dyes, dansyl chloride, phycoerythrin, phycocyanin, fluorescent lanthanide complexes such as those including Europium and Terbium, Cy3, Cy5, Cy7, Alexa Fluor® dyes and others known in the art such as those described in Principles of Fluorescence Spectroscopy, Joseph R. Lakowicz (Editor), Plenum Pub Corp, 2nd edition (July 1999) and the 6th Edition of Molecular Probes Handbook by Richard P. Hoagland.

A secondary label can be useful. A secondary label is a binding moiety that can bind specifically to a partner moiety. For example, a ligand moiety can be attached to a polymerase, nucleic acid or nucleotide to allow detection via specific affinity of the ligand for a detectable receptor, such as a labeled receptor. Exemplary pairs of binding moieties that can be used include, without limitation, antigen and immunoglobulin or active fragments thereof, such as a single-chain variable fragments (scFv) or antigen binding fragment (Fab); immunoglobulin and immunoglobulin (or active fragments, respectively); avidin and biotin, or analogs thereof having specificity for avidin; streptavidin and biotin, or analogs thereof having specificity for streptavidin; complementary oligonucleotides; or carbohydrates and lectins.

In some embodiments, the secondary label can be a chemically modifiable moiety. In this embodiment, labels having reactive functional moieties can be incorporated into a stabilized ternary complex. Subsequently, the functional moiety can be covalently reacted with a primary label moiety. Suitable functional moieties include, but are not limited to, amino moieties, carboxy moieties, maleimide moieties, oxo moieties and thiol moieties.

In some embodiments, a polymerase-nucleic acid complex, or components that participate in forming such a complex, can lack exogenous labels. For example, a ternary complex and all components participating in the ternary complex (e.g., polymerase, template nucleic acid, primer and/or cognate nucleotide) can lack one, several or all of the exogenous labels described herein or in the above-incorporated references. In such embodiments, ternary complexes (e.g., stabilized ternary complexes) can be detected based on intrinsic properties of the ternary complex, such as mass, charge, intrinsic optical properties or the like. Exemplary methods for detecting non-labeled ternary complexes are set forth in U.S. Pat. App. Pub. Nos. 2017/0022553 A1 published on Jan. 26, 2017 and 2018/0044727 A1 published on Feb. 15, 2018; and WO 2017/117243 published on Jul. 6, 2017, each of which is incorporated herein by reference.

A stabilized ternary complex, or a component that is capable of participating in the formation of a ternary complex, can be attached to a surface such as a surface in or on a solid support. The solid support can be made from any of a variety of materials used for analytical biochemistry. Suitable materials may include glass, polymeric materials, silicon, quartz (fused silica), borofloat glass, silica, silica-based materials, carbon, metals, an optical fiber or bundle of optical fibers, sapphire, or plastic materials. The material can be selected based on properties desired for a particular use. For example, materials that are transparent to a desired wavelength of radiation are useful for analytical techniques that will utilize radiation of that wavelength. Conversely, it may be desirable to select a material that does not pass radiation of a certain wavelength (e.g., being opaque, absorptive or reflective). Other properties of a material that can be exploited are inertness or reactivity to certain reagents used in a particular method, ease of manipulation, or low cost of manufacture.

A particularly useful solid support is a particle such as a bead or microsphere. Populations of beads can be used for attachment of populations of polymerase-nucleic acid complexes or components capable of forming the complexes (e.g., polymerases, templates, primers or nucleotides). It may be useful to use a configuration whereby each bead has a single type of complex or a single type of component capable of forming a complex. For example, an individual bead can be attached to a single type of binary complex, a single type of ternary complex, a single type of primed-template nucleic acid, a single type of primer, a single type of template, a single type of polymerase or a single type of nucleotide. In some embodiments, different types of components need not be separated on a bead-by-bead basis. As such, a single bead can bear multiple different types of polymerase-nucleic acid complexes, template nucleic acids, primers, primed-template nucleic acids and/or nucleotides.

The composition of a bead can vary, depending for example, on the format, chemistry and/or method of attachment to be used. Exemplary bead compositions include solid supports, and chemical functionalities imparted thereto, used in protein and nucleic acid capture methods. Such compositions include, for example, plastics, ceramics, glass, polystyrene, melamine, methylstyrene, acrylic polymers, paramagnetic materials, thoria sol, carbon graphite, titanium dioxide, latex or cross-linked dextrans such as Sepharose™, cellulose, nylon, cross-linked micelles and Teflon™, as well as other materials set forth in “Microsphere Detection Guide” from Bangs Laboratories, Fishers Ind., which is incorporated herein by reference.

In some embodiments, beads can be arrayed or otherwise spatially distinguished. Exemplary bead-based arrays that can be used include, without limitation, a BeadChip™ Array available from Illumina, Inc. (San Diego, Calif.) or arrays such as those described in U.S. Pat. Nos. 6,266,459; 6,355,431; 6,770,441; 6,859,570; and 7,622,294; or PCT Publication No. WO 00/63437 published on Oct. 26, 2000, each of which is incorporated herein by reference. Beads can be located at discrete locations, such as wells, on a solid support, whereby each location accommodates a single bead. Alternatively, discrete locations where beads reside can each include a plurality of beads as described, for example, in U.S. Pat. App. Pub. Nos. 2004/0263923 A1 published Dec. 30, 2004, 2004/0233485 A1 published on Nov. 25, 2004, 2004/0132205 A1 published on Jul. 8, 2004, and 2004/0125424 A1 published on Jul. 1, 2004, each of which is incorporated herein by reference.

As will be recognized from the above bead array embodiments, a method of the present disclosure can be carried out in a multiplex format whereby multiple different analytes are manipulated in parallel. The manipulations can be, for example, detection of the analytes, synthesis of the analytes, modification of the analytes, dissociation of complexed analytes, association of analytes into a complex or the like. Although it is also possible to serially manipulate different types of analytes using one or more steps of the methods set forth herein, parallel processing can provide cost savings, time savings and uniformity of conditions. An apparatus or method of the present disclosure can include at least 2, 10, 100, 1×10³, 1×10⁴, 1×10⁵, 1×10⁶, 1×10⁹, or more different analytes. Alternatively or additionally, an apparatus or method of the present disclosure can include at most 1×10⁹, 1×10⁶, 1×10⁵, 1×10⁴, 1×10³, 100, 10, 2 or fewer, different analytes. Any of a variety of analytes can be used such as polymerase-nucleic acid complexes or components that participate in formation of the complexes. Accordingly, various reagents or products set forth herein as being useful in the apparatus or methods (e.g., primed-template nucleic acids, polymerases, nucleotides or stabilized ternary complexes) can be multiplexed to have different types or species in these ranges. The different analytes that are present in an array can be located at different features of the array. Thus, signals acquired from a feature will be indicative of a particular analyte (e.g., a different nucleic acid sequence) present at the feature.

Further examples of commercially available arrays that can be used include, for example, an Affymetrix GeneChip™ array. A spotted array can also be used according to some embodiments. An exemplary spotted array is a CodeLink™ Array commercialized by from Amersham Biosciences. Another array that is useful is one that is manufactured using inkjet printing methods such as SurePrint™ Technology commercialized by Agilent Technologies.

Other useful arrays include those that are used in nucleic acid sequencing applications. For example, arrays that are used to attach amplicons of genomic fragments (often referred to as clusters) can be particularly useful. Examples of nucleic acid sequencing arrays that can be used herein include those described in Bentley et al., Nature 456:53-59 (2008), PCT Pub. Nos. WO 91/06678 published on May 16, 1991; WO 04/018497 published Mar. 4, 2004 and WO 07/123744 published on Nov. 11, 2007; U.S. Pat. Nos. 7,057,026; 7,211,414; 7,315,019; 7,329,492 and 7,405,281; or U.S. Pat. App. Pub. No. 2008/0108082 published on May 8, 2008, each of which is incorporated herein by reference.

An analyte, such as a nucleic acid, can be attached to a support in a way that provides detection of the analyte at a single molecule level or at an ensemble level. For example, a plurality of different nucleic acids can be attached to a solid support in a way that an individual stabilized ternary complex that forms on one nucleic acid molecule on the support can be distinguished from all neighboring ternary complexes that form on the nucleic acid molecules of the support. As such, one or more different nucleic acids can be attached to a solid support in a format where each single nucleic acid molecule is physically isolated and detected in a way that the single nucleic acid molecule is resolved from all other nucleic acid molecules on the solid support. Similarly, one or more different polymerase-nucleic acid complexes can be attached to a solid support in a format where each single polymerase-nucleic acid complex is physically isolated and detected in a way that the single polymerase-nucleic acid complex is resolved from all other polymerase-nucleic acid complexes on the solid support.

In some embodiments, a method of the present disclosure can be carried out for one or more nucleic acid ensembles, a nucleic acid ensemble being a population of nucleic acids having a common template sequence. An ensemble can include, for example, at least 2, 10, 50, 100, 500, 1000 or more nucleic acids having a common template sequence. Alternatively or additionally, an ensemble can include at most, at most about, at least, at least about, or 1000, 500, 100, 50, 10, 2, or a range between any two of these values, nucleic acids having a common template sequence. An ensemble that is present at a feature of an array can be clonal such that substantially all of the nucleic acids at the feature have a common template sequence. However, a feature need not contain a clonal population of nucleic acids. Rather, a feature can include a mixed population of nucleic acids, wherein a particular template sequence is present in a majority of the nucleic acids. For example, a population of nucleic acids that is at a particular feature can include at least 51%, 60%, 75%, 90%, 95% or 99% or more species having a particular template sequence. A feature having a non-clonal population of nucleic acids can be detected under conditions that allow the population to be detected as an ensemble, whereby the total signal acquired from the feature represents an average of signals produced by the non-clonal population. So long as contaminating nucleic acids are present as a minority at a feature of interest, the average signal can provide a means to characterize the majority of template nucleic acids at the feature.

Cluster methods can be used to attach one or more ensembles to a solid support. As such, an array can have a plurality of ensembles, each of the ensembles being referred to as a cluster or array feature in that format. Clusters can be formed using methods known in the art such as bridge amplification or emulsion PCR. Useful bridge amplification methods are described, for example, in U.S. Pat. Nos. 5,641,658 and 7,115,400; U.S. Patent Pub. Nos. 2002/0055100 A1 published on May 9, 2002; 2004/0002090 A1 published on Jan. 1, 2004; 2004/0096853 A1 published on May 20, 2004; 2007/0128624 A1 published on Jun. 7, 2007; and 2008/0009420 Al published on January 10, 2008. Emulsion PCR methods include, for example, methods described in Dressman et al., Proc. Natl. Acad. Sci. USA 100:8817-8822 (2003), WO 05/010145, or U.S. Patent Pub. Nos. 2005/0130173 A1 published on Jun. 16, 2005 and 2005/0064460 A1 published on Mar. 24, 2005, each of which is incorporated herein by reference in its entirety. Another useful method for amplifying nucleic acids on a surface is rolling circle amplification (RCA), for example, as described in Lizardi et al., Nat. Genet. 19:225-232 (1998) or US 2007/0099208 A1 published on May 3, 2007, each of which is incorporated herein by reference.

In some embodiments, one or more of the polymerase-nucleic acid complex, polymerase, primer, template, primed-template nucleic acid, and nucleotide is attached to a flow cell surface or to a solid support in a flow cell. A flow cell allows convenient fluidic manipulation by passing solutions into and out of a fluidic chamber that contacts the support-bound analyte. The flow cell also provides for detection of the fluidically manipulated analyte(s). For example, a detector can be positioned to detect signals from the solid support, such as signals from a label that is recruited to the solid support due to formation of a stabilized ternary complex. Exemplary flow cells that can be used are described, for example, in US Pat. App. Pub. No. 2010/0111768 A1 or 2012/0270305 A1, or WO 05/065814, each of which is incorporated herein by reference.

In some embodiments, only one of the components of a polymerase-nucleic acid complex (e.g., a ternary complex) is independently immobilized. In some embodiments, two or more of the components of a polymerase-nucleic acid complex (e.g., a ternary complex) are independently immobilized. For example, a primed-template nucleic acid can be independently immobilized on a solid support such that it remains immobilized independent of being associated with other components of the polymerase-nucleic acid complex. Similarly, a polymerase can be independently immobilized on a solid support such that it remains immobilized independent of being associated with other components of the polymerase-nucleic acid complex. Immobilization can be mediated by chemistries that are used to attach analytes to arrays as set forth herein or in references cited in connection with arrays herein. In such embodiments, a ternary complex can be dissociated using reagents set forth herein that selectively dissociate the nucleotide from the polymerase and primed-template nucleic acid while maintaining association between the polymerase and primed-template nucleic acid. This association can be exploited to retain the polymerase and primed-template nucleic acid so long as one of the pair is independently immobilized to a solid support. The nucleotide can then be removed by separating the fluid containing nucleotide from the solid support.

In some embodiments, a surface-immobilized polymerase-nucleic acid complex is covalently attached to the surface. For example, the polymerase of the surface-immobilized polymerase-nucleic acid complex can be covalently attached to the surface. Alternatively or additionally, the primed-template nucleic acid of the surface-immobilized polymerase-nucleic acid complex can be covalently attached to the surface. Alternatively or additionally, a surface-immobilized polymerase-nucleic acid complex is non-covalently attached to the surface. For example, attachment can result from the binding of a ligand to a receptor, wherein the ligand is attached to the polymerase-nucleic acid complex and the receptor is attached to the surface, or vice versa. When used in methods that include a step of dissociating a nucleotide from a polymerase-nucleic acid complex, the means of attachment can be inert to the reagents and conditions used for dissociation. In this way, the polymerase-nucleic acid complex can be retained upon dissociation of the nucleotide.

Any of a variety of vessels can be used for a method or composition set forth herein. For example, the vessel can be selected from the group consisting of a flow cell, a well in a multi-well plate, a droplet, a vesicle, a test tube, a tray, a centrifuge tube, tubing and a channel in a substrate. Other vessels known in the art of molecular biology, biochemistry, analytical chemistry or other relevant arts can be used as deemed appropriate.

In some embodiments of the compositions and methods set forth herein, a polymerase-nucleic acid complex, or at least one of the components that is capable of forming the complex, can be in contact with an aqueous solution that includes a polyol, diol, sulfone or sulfoxide. The aqueous solution can be composed of at least 25% water on a volume to volume (v/v) basis with respect to other solvent(s) in the solution. For example, the aqueous solution can be composed of at least 35%, 51%, 75%, 90%, 95%, 99% or more (v/v) of water. Generally, the aqueous solution will be a single-phase solution. However, multiphase solutions such as a foam (gas bubbles in an aqueous solution), emulsion (water-immiscible solvent droplets in an aqueous solution) or slurry (solid-phase particles in an aqueous solution) can be useful in some methods and compositions set forth herein. See for example, U.S. patent application Ser. No. 16/700,422 published as US20200171498 on Jun. 4, 2020 and US Pat. App. Pub. No. 2019/0119740 A1 published on Apr. 25, 2019, each of which is incorporated herein by reference.

An aqueous solution can contain one or more agents that function to modify a ternary complex (i.e., a complex between a primed-template, polymerase and nucleotide) by dissociating the nucleotide from the complex without dissociating the polymerase from the primed-template. Useful agents include a polyol, diol, sulfone, sulfoxide, and any combinations thereof.

Useful polyols include, for example, low molecular weight polyols such as diols set forth herein or polymeric diols such as polyethylene glycol (PEG) and polyvinyl alcohol (PVA). Useful polyols can have a molecular weight that is at most 100 kiloDaltons (kDa), 50 kDa, 30 kDa, 20 kDa, 10 kDa or less. Alternatively or additionally, the polyol can have a molecular weight that is at least 10 kDa, 20 kDa, 30 kDa, 50 kDa, 100 kDa or more.

An aqueous solution that is used in a method or composition herein can contain polyol in an amount that is at least about 0.5%, 1%, 5%, 10%, 15%, 20%, 25%, 30%, 35%, 40%, 45%, 50% or more of the polyol. Alternatively or additionally, the aqueous solution can contain at most about 50%, 45%, 40%, 35%, 30%, 25%, 20%, 15%, 10%, 5%, 1%, 0.5% or less of the polyol.

Useful diols include, for example, aliphatic diols, saturated aliphatic diols, non-saturated aliphatic diols, aromatic diols, geminal diols, vicinal diols or terminal diols. Optionally, a diol can have a linear carbon chain. Optionally, a diol can have a non-branched carbon chain. Optionally, a diol can have a branched carbon chain. Diols having hydrocarbon chains can include a linear or branched chain having at least 2, 3, 4, 5, 6, 7, 8 or more carbons. Alternatively or additionally, a linear or branched hydrocarbon chain of a diol can include at most 8, 7, 6, 5, 4, 3, or 2 carbons.

Particularly useful aliphatic diols include, for example, propylene glycol, 1,3 butane diol, 1,5 pentanediol, 1,6 hexanediol and 1,7 heptanediol.

An aqueous solution that is used in a method or composition herein can contain a diol in an amount that is at least about 0.5%, 1%, 5%, 10%, 15%, 20%, 25%, 30%, 35%, 40%, 45%, 50% or more of the diol. Alternatively or additionally, an aqueous solution can contain at most about 50%, 45%, 40%, 35%, 30%, 25%, 20%, 15%, 10%, 5%, 1%, 0.5% or less of the diol.

Useful sulfoxides include, for example, a sulfone. Exemplary, sulfoxides include, but are not limited to dimethyl sulfoxide, ethyl methyl sulfone and sulfolane.

An aqueous solution that is used in a method or composition herein can contain sulfoxide in an amount that is at least about 0.5%, 1%, 5%, 10%, 15%, 20%, 25%, 30%, 35%, 40%, 45%, 50% or more of the sulfoxide. Alternatively or additionally, the aqueous solution can contain at most about 50%, 45%, 40%, 35%, 30%, 25%, 20%, 15%, 10%, 5%, 1%, 0.5% or less of the sulfoxide.

Generally, an aqueous solution will contain a polyol, diol, sulfone or sulfoxide that is miscible in water or present in an amount that is soluble in the aqueous solution. In some embodiments, salt and organic solvent (e.g., alcohol) are both present, for example, each in an amount set forth herein.

A further useful chemical condition for dissociating a nucleotide from a ternary complex is pH outside of the physiological range (e.g., at or below pH 6, 5, or 4; at or above pH 8, 9 or 10). Other reagents that can be useful include, but are not limited to, redox reagents such as dithiothreitol, glutathione or 2-mercaptoethanol; detergents such as anionic, cationic or zwitterionic detergents; or proteins that bind to nucleotides (e.g., proteins that compete with polymerase for binding to nucleotides). The chemical conditions set forth herein for dissociating nucleotide from a ternary complex can be used in various combinations (e.g., an aqueous solution can have a pH inside or outside of physiological range and can also include a miscible organic solvent). As a further option, one or more chemical condition for dissociating nucleotide from a ternary complex can be combined with a physical condition for dissociating nucleotide from a ternary complex.

An aqueous solution that contains a polyol, diol, sulfone, sulfoxide, or a combination thereof, can further contain Lithium and/or Betaine. In some embodiments, the aqueous solution contain Lithium. Lithium can be at a concentration of at least 5 mM, 10 mM, 25 mM, 50 mM, 100 mM, 250 mM or higher. Alternatively or additionally, Lithium can be present at a concentration of at most 250 mM, 100 mM, 50 mM, 25 mM, 10 mM, 5 mM or less. In some embodiments, the aqueous solution contain Betaine. Betaine can be present at a concentration of at least 1 mM, 10 mM, 50 mM, 100 mM, 500 mM, 1 M, 2 M, 3 M, 3.5M or higher. Alternatively or additionally, Betaine can be present at a concentration of at most 3.5 M, 2 M, 1 M, 500 mM, 100 mM, 50 mM, 10 mM, 1 mM, or less. In some embodiments, the aqueous solution contain Lithium and Betaine. Lithium or Betaine can be used, for example, as set forth in US Pat. No. 10,400,272, which is incorporated herein by reference.

Particularly useful polyols, diols, sulfone or sulfoxides are those that are non-flammable. Accordingly, the agent itself or an aqueous solution containing the agent can have a flash point above 100° F. (38° C.). Alternatively or additionally, the agent or the aqueous solution that contains the agent can have a flammability rating of 0, 1 or 2 in the Hazardous Materials Identification System (HMIS) used by regulatory bodies such as those under the jurisdiction of the United States Government.

Another characteristic that can be useful is the ability for a polyol, diol, sulfone or sulfoxide to maintain a fluid foam. Accordingly, an aqueous solution that contains a polyol, diol, sulfone or sulfoxide can be capable of maintaining a minimum number of bubbles in a foam, a minimum size for bubbles in a foam, a maximum size for bubbles in a foam or the like. For example, the number of bubbles can be quantified as a concentration (i.e., volume fraction) of bubbles in a fluid foam and the concentration can be at least 0.01%, 0.1%, 1%, 5%, 10%, 25%, 50%, 75%, 90% 95%, 99% or more of the total volume of the fluid foam in a particular vessel. Regarding bubble size, an aqueous solution that contains polyol, diol, sulfone or sulfoxide can support bubbles having an effective diameter that is larger than 10 nm, 50 nm, 100 nm, 1 μm, 10 μm, 100 μm, 500 μm or larger. Alternatively or additionally, an aqueous solution that contains polyol, diol, sulfone or sulfoxide can support bubbles having an effective diameter that is smaller than 500 μm, 100 μm, 10 μm, 1 μm, 100 nm, 50 nm, 10 nm or smaller. Optionally, an aqueous solution that contains polyol, diol, sulfone or sulfoxide can support bubbles having an effective diameter that is smaller than the diameter of a channel or other vessel where the bubbles reside. The number and or size of droplets in an emulsion can be in a range set forth herein for bubbles in a foam. Foams and emulsions can be made and used, for example, as set forth in U.S. patent application Ser. No. 16/700,422 published as US20200171498 on Jun. 4, 2020, which is incorporated herein by reference.

It will be understood that a composition or method of the present disclosure can be configured to include a combination of two or more of the polyols, diols, sulfones or sulfoxides set forth herein. A combination can be useful, for example, when one compound is desirable for removing one type of nucleotide from a polymerase-nucleic acid complex and a second type of compound is desirable for removing a second type of nucleotide from a polymerase-nucleic acid complex.

A combination of compounds can contain two or more compounds that share a particular characteristic such as being the same type of compound. For example, a combination can include two or more polyols, two or more triols, two or more tetrols, two or more diols, two or more aliphatic diols, two or more aromatic diols, two or more terminal diols, two or more diols having four to seven carbons, two or more diols having five to seven carbons, two or more sulfoxides, two or more sulfones etc. In some embodiments, a combination can include two or more compounds having different characteristics such as being different types of compounds. For example, a combination can include a diol and at least one of a sulfoxide, sulfone, polyol or other type of compound set forth herein; an aliphatic diol and at least one of a sulfoxide, sulfone, polyol or other type of compound set forth herein; a terminal diol and at least one of a sulfoxide, sulfone, polyol or other type of compound set forth herein; a diol having a carbon chain with four to seven carbons and at least one of a sulfoxide, sulfone, polyol or other type of compound set forth herein; a diol having a carbon chain with five to seven carbons and at least one of a sulfoxide, sulfone, polyol or other type of compound set forth herein; etc.

Accordingly, an aqueous solution that is used in a method or composition herein can contain two or more compounds the combination of which amounts to at least about 0.5%, 1%, 5%, 10%, 15%, 20%, 25%, 30%, 35%, 40%, 45%, 50% or more of the aqueous solution. Alternatively or additionally, the aqueous solution that is used in a method or composition herein can contain two or more compounds the combination of which amounts to at most about 50%, 45%, 40%, 35%, 30%, 25%, 20%, 15%, 10%, 5%, 1%, or 0.5% of the aqueous solution.

It will be understood that a composition or method of the present disclosure can be configured to lack polyols, diols, sulfones or sulfoxides, including but not limited to particular types of polyols, diols, sulfones or sulfoxides set forth herein; polyols, diols, sulfones or sulfoxides having particular characteristics set forth herein; or particular polyol species, diol species, sulfone species or sulfoxide species set forth herein.

The present disclosure provides a method for modifying polymerase-nucleic acid complexes. The method can include step (a) providing a plurality of polymerase-nucleic acid complexes each comprises a polymerase and a primed-template nucleic acid, wherein at least a subset of the polymerase-nucleic acid complexes are ternary complexes further comprising nucleotides; and (b) contacting the plurality of polymerase-nucleic acid complexes with an aqueous solution comprising a polyol, alcohol, aliphatic diol, sulfone, sulfoxide, or a combination thereof, thereby dissociating the nucleotides from the subset of polymerase-nucleic acid complexes. The aqueous solution can, for example, further comprises Lithium, Betaine, or both. The plurality of polymerase-nucleic acid complexes can, in some embodiments, be immobilized on a surface, present in a vessel, or both. Step (b) contacting the plurality of polymerase-nucleic acid complexes with the aqueous solution can comprise washing the surface on which the plurality polymerase-nucleic acid complexes are immobilized with the aqueous solution, thereby removing the nucleotides from the subset of polymerase-nucleic acid complexes that are ternary complexes in the vessel. The method, in some embodiments, can include the steps of (a) providing a plurality of surface-immobilized polymerase-nucleic acid complexes in a vessel, wherein the nucleic acid includes a primed-template nucleic acid, wherein at least a subset of the surface-immobilized polymerase-nucleic acid complexes includes ternary complexes further comprising nucleotides; and (b) washing the surface with an aqueous solution comprising a polyol, diol, sulfone or sulfoxide, thereby removing the nucleotides from the vessel and retaining the surface-immobilized polymerase-nucleic acid complexes in the vessel.

As described herein, it is advantageous in some instances to dissociate a nucleotide from a ternary complex, thereby allowing the nucleotide to be separated from the primed-template nucleic acid without causing substantial removal of the polymerase which is expensive and time consuming to produce and to be delivered to the primed-template nucleic acid. The methods and compositions disclosed herein enable such saving in time and resources. For example, using the methods and compositions disclosed herein, after contacting the plurality of polymerase-nucleic acid complexes with the aqueous solution, 10%, 15%, 20%, 25%, 30%, 35%, 40%, 45%, 50%, 55%, 60%, 65%, 70%, 75%, 80%, 85%, 90%, 95%, 96%, 97%, 98%, 99%, 100%, or a number or a range between any two of these numbers, of the polymerases can be retained in the plurality of polymerase-nucleic acid complexes. Using the methods and compositions disclosed herein, after contacting the plurality of polymerase-nucleic acid complexes with the aqueous solution, at least or at least about 10%, 15%, 20%, 25%, 30%, 35%, 40%, 45%, 50%, 55%, 60%, 65%, 70%, 75%, 80%, 85%, 90%, 95%, 96%, 97%, 98%, 99% of the polymerases can be retained in the plurality of polymerase-nucleic acid complexes. In some embodiments, after contacting the plurality of polymerase-nucleic acid complexes with the aqueous solution, at least 50% of the polymerases are retained in the plurality of polymerase-nucleic acid complexes. In some embodiments, after contacting the plurality of polymerase-nucleic acid complexes with the aqueous solution, at least 70% of the polymerases are retained in the plurality of polymerase-nucleic acid complexes. For example, using the methods and compositions disclosed herein, after contacting the plurality of polymerase-nucleic acid complexes with the aqueous solution, 5%, 10%, 15%, 20%, 25%, 30%, 35%, 40%, 45%, or 50% of the polymerases can dissociate from the plurality of polymerase-nucleic acid complexes. Using the methods and compositions disclosed herein, after contacting the plurality of polymerase-nucleic acid complexes with the aqueous solution, at most or at most about 5%, 10%, 15%, 20%, 25%, 30%, 35%, 40%, 45%, or 50% of the polymerases dissociate from the plurality of polymerase-nucleic acid complexes. In some embodiments, at most 5% of the polymerases dissociate from the plurality of polymerase-nucleic acid complexes. In some embodiments, at most 20% of the polymerases dissociate from the plurality of polymerase-nucleic acid complexes.

Using the methods and compositions disclosed herein, after contacting the plurality of polymerase-nucleic acid complexes with the aqueous solution, 40%, 45%, 50%, 55%, 60%, 65%, 70%, 75%, 80%, 85%, 90%, 95%, 96%, 97%, 98%, 99%, 100%, or a number or a range between any two of these numbers, of the nucleotides from the ternary complexes can be dissociated from the ternary complexes. Using the methods and compositions disclosed herein, after contacting the plurality of polymerase-nucleic acid complexes with the aqueous solution, at least or at least about 40%, 45%, 50%, 55%, 60%, 65%, 70%, 75%, 80%, 85%, 90%, 95%, 96%, 97%, 98%, 99%, 100%, or a number or a range between any two of these numbers, of the nucleotides from the ternary complexes can be dissociated from the ternary complexes. In some embodiments, at least 50% of the nucleotides are dissociated from the ternary complex. In some embodiments, at least 70% of the nucleotides are dissociated from the ternary complex. It can be advantageous to avoid substantial retention of the nucleotides in the ternary complex. For example, using the methods and compositions disclosed herein, after contacting the plurality of polymerase-nucleic acid complexes with the aqueous solution, 0.5%, 1%, 2%, 3%, 4%, 5%, 10%, 15%, 20%, 25%, 30%, 35%, 40%, or a number or a range between any two of these numbers, of the nucleotides from the ternary complexes can be dissociated from the ternary complexes. Using the methods and compositions disclosed herein, after contacting the plurality of polymerase-nucleic acid complexes with the aqueous solution, at most or at most about, 0.5%, 1%, 2%, 3%, 4%, 5%, 10%, 15%, 20%, 25%, 30%, 35%, 40% of the nucleotides from the ternary complexes can be dissociated from the ternary complexes.

A plurality of surface-immobilized polymerase-nucleic acid complexes can be provided in any of a variety of ways. For example, the complexes may have been formed and/or immobilized prior to performing the method for their modification. The complexes may have been produced at another location and shipped to an individual who uses the complexes, for example, to perform a method set forth herein. Accordingly, a plurality of surface-immobilized polymerase-nucleic acid complexes can be retrieved from a storage location (e.g., a refrigerated or frozen storage location) prior to use in a method set forth herein.

In some embodiments, the plurality of surface-immobilized polymerase-nucleic acid complexes can be prepared as part of a method set forth herein. In this regard, a method for modifying polymerase-nucleic acid complexes can include the steps of (a) providing a plurality of surface-immobilized polymerase-nucleic acid complexes in a vessel, wherein the nucleic acid includes a primed-template nucleic acid, wherein at least a subset of the surface-immobilized polymerase-nucleic acid complexes includes ternary complexes further comprising nucleotides; and (b) washing the surface with an aqueous solution comprising a polyol, diol, sulfone or sulfoxide, thereby removing the nucleotides from the vessel and retaining the surface-immobilized polymerase-nucleic acid complexes in the vessel, wherein step (a) includes contacting a plurality of polymerases with a plurality of primed-template nucleic acids and with a plurality of nucleotides, thereby providing the plurality of surface-immobilized polymerase-nucleic acid complexes in the vessel, each of the surface-immobilized polymerase-nucleic acid complexes including a polymerase of the plurality of polymerases and a primed-template nucleic acid of the plurality of primed-template nucleic acids, the nucleotides in the ternary complexes being nucleotides of the plurality of nucleotides.

In some embodiments of the above method (or other methods set forth herein), a plurality of polymerases can be attached to a surface prior to being contacted with a plurality of primed-template nucleic acids. Alternatively, a plurality of primed-template nucleic acids can be attached to a surface prior to being contacted with a plurality of polymerases. Generally, only one of the polymerase and the nucleic acid is surface-immobilized prior to binding together to form a polymerase-nucleic acid complex. As such, the second component becomes immobilized by virtue of the binding event that produces the surface-immobilized polymerase-nucleic acid complex.

In some embodiments of the above method (or other methods set forth herein), a plurality of surface-immobilized polymerase-nucleic acid complexes is formed by simultaneously contacting a plurality of polymerases with a plurality of primed-template nucleic acids and with a plurality of nucleotides. Alternatively, a plurality of surface-immobilized polymerase-nucleic acid complexes is formed by sequentially contacting a plurality of polymerases with a plurality of primed-template nucleic acids and with a plurality of nucleotides. For example, the plurality of primed-template nucleic acids can be initially contacted with the plurality of polymerases and then with the plurality of nucleotides. In another example, the plurality of polymerases can be initially contacted with the plurality of nucleotides and then with the plurality of primed-template nucleic acids.

Optionally, one or more different types of nucleotides can be contacted with a polymerase, nucleic acid (e.g., a primed template nucleic acid) or polymerase-nucleic acid complex. For example, at least 1, 2, 3, 4 or more nucleotide types can be used. Alternatively or additionally, at most 4, 3, 2, or 1 nucleotide types can be used. The nucleotide types can differ due to the structural characteristics that determine Watson-Crick base pairing. Accordingly, one or more nucleotide types that are present can be cognates for at least 1, 2, 3 or 4 base types in a template nucleic acid that is used in a method set forth herein. Alternatively or additionally, one or more nucleotide types that are present can be cognates for at most 4, 3, 2, or 1 base types in a template nucleic acid that is used in a method set forth herein. Other structural and functional characteristics that can differ between nucleotide types include, but are not limited to, the presence or absence of label moieties, the structure of label moieties attached to the respective nucleotides, the detectable characteristics of label moieties attached to the respective nucleotides, the presence or absence of blocking moieties, the structure of blocking moieties attached to the respective nucleotides, or the reactivity of blocking moieties attached to the respective nucleotides. It will be understood that two nucleotide types can differ based on one or a combination of these characteristics.

For ease of explanation, methods of the present disclosure are exemplified herein with respect to stabilized ternary complex(es) formed in the presence of nucleotide cognate for one base type. It will be understood, that ternary complex(es) can be formed in the presence of nucleotide cognate(s) for only one base type, for example, in the presence of only a single type of nucleotide or in the presence of multiple nucleotide types that are cognates for the same base type. Alternatively, ternary complex(es) can be formed in the presence of a mixture of nucleotide types that are cognates for more than one base type expected to be in a template nucleic acid. For example, the nucleotide types that are present during a particular step of the methods set forth herein can be cognates for at least 2, 3 or 4 different base types expected to be in a template nucleic acid. Alternatively or additionally, the nucleotide types that are present in a particular step of the methods set forth herein can be cognates for at most 4, 3 or 2 different base types. The different nucleotide types can be mixed with each other prior to being delivered to a vessel where a primed-template nucleic acid occurs. In other embodiments, different nucleotide types can be serially delivered to a vessel where a primed-template nucleic acid occurs. As such, the different nucleotides will accumulate to create a reaction mixture where the different types of nucleotides are simultaneously present with the primed-template nucleic acid.

A method of the present disclosure can include a wash step in which one or more analyte or reagent is removed from a solid support and/or from a vessel. The wash solution can contain a diol, polyol, sulfone or sulfoxide. In some embodiments, the analyte is a nucleotide that is dissociated from a ternary complex. The nucleotide can be removed from a vessel under conditions that will dissociate the nucleotide from a ternary complex, thereby allowing the nucleotide to be separated from the primed-template nucleic acid without causing substantial removal of the polymerase from the template nucleic acid. For example, the dissociated nucleotide can be removed via flow of fluid away from the primed-template nucleic acid (e.g., through a flow cell), decanting fluid away from the primed-template nucleic acid, separating a solid support that is attached to the primed-template nucleic acid from the fluid (e.g., via magnetic or gravity based separation of particles that are attached to the primed-template nucleic acid), etc. Another nucleotide can then be delivered to the primed-template nucleic acid. This other nucleotide can be, but need not necessarily be, a different type of nucleotide from the one that was previously removed. Delivery of more polymerase is not necessary if the polymerase is not substantially removed from the presence of the primed-template nucleic acid. This provides a savings of time and resources that would otherwise be spent preparing more polymerase.

A method of the present disclosure can include a step of detecting a polymerase-nucleic acid complex such as a ternary complex (e.g., a stabilized ternary complex). Accordingly, a method for modifying polymerase-nucleic acid complexes can include the steps of (a) providing a plurality of surface-immobilized polymerase-nucleic acid complexes in a vessel, wherein the nucleic acid includes a primed-template nucleic acid, wherein at least a subset of the surface-immobilized polymerase-nucleic acid complexes includes ternary complexes further comprising nucleotides; (b) detecting the surface-immobilized polymerase-nucleic acid complexes; and (c) washing the surface with an aqueous solution comprising a polyol, diol, sulfone or sulfoxide, thereby removing the nucleotides from the vessel and retaining the surface-immobilized polymerase-nucleic acid complexes in the vessel.

Detection of a polymerase-nucleic acid complex, whether immobilized or in solution phase, can be facilitated by exogenous labels attached to a nucleotide, polymerase, or nucleic acid. Signals produced by the exogenous labels can be detected to determine the presence of the polymerase-nucleic acid complex that contains the exogenous label. In some embodiments, detection can be performed to acquire signals that distinguish at least 2, 3, 4 or more types of nucleotides in the ternary complexes. In some embodiments, detection can be performed to acquire signals that distinguish nucleotide cognates for at least 2, 3, 4 or more types of bases known or suspected of being in a template nucleic acid.

A method of the present disclosure can be performed in a mode whereby different nucleotide types are serially delivered and then removed from a vessel where ternary complex is to be formed and examined. In this mode, a first nucleotide type can be delivered to a polymerase-nucleic acid complex in a vessel and then the first nucleotide type can be removed from the vessel prior to delivering a second nucleotide type to the vessel. Polymerase can be retained in the vessel upon removal of the first nucleotide. As such, polymerase can be delivered to a flow cell initially to create conditions that facilitate ternary complex formation with the first nucleotide and new polymerase can be, but need not be, added in a subsequent delivery to facilitate ternary complex formation with nucleotides that are subsequently delivered.

Accordingly, a method for modifying polymerase-nucleic acid complexes can include the steps of (a) contacting a plurality of polymerases with a plurality of primed-template nucleic acids and with a plurality of nucleotides, thereby providing a plurality of surface-immobilized polymerase-nucleic acid complexes in the vessel, each of the surface-immobilized polymerase-nucleic acid complexes including a polymerase of the plurality of polymerases and a primed-template nucleic acid of the plurality of primed-template nucleic acids, the nucleotides in the ternary complexes including nucleotides of the plurality of nucleotides; (b) washing the surface with an aqueous solution comprising a polyol, diol, sulfone or sulfoxide, thereby removing the nucleotides from the vessel and retaining the surface-immobilized polymerase-nucleic acid complexes in the vessel; and (c) delivering a solution including a plurality of second nucleotides to the vessel, whereby at least a second subset of the surface-immobilized polymerase-nucleic acid complexes includes ternary complexes further including second nucleotides from the plurality of second nucleotides.

Optionally, the second nucleotides used in the above method are a different type of nucleotide compared to the nucleotides. However, it will be understood that the second nucleotides can include at least one nucleotide type that is the same as a nucleotide type in the plurality of nucleotides. Optionally, the above method can further include a step of detecting the ternary complexes that further include the second nucleotides. For example, the second nucleotides can have exogenous labels and the exogenous labels of the second nucleotides can be detected in the ternary complexes that further have the second nucleotides. Optionally, the ternary complexes that further have the second nucleotides can be detected by acquiring signals that distinguish at least 2, 3 or 4 types of the nucleotides.

When performing a method of the present disclosure in a mode whereby different nucleotide types are serially delivered to a reaction vessel and then removed from the vessel, examination of the vessel for ternary complexes can be carried out after each delivery. In this mode, ternary complexes of different types (i.e., ternary complexes that differ in the type of nucleotide that is present) will form after each delivery. Ternary complexes that had formed in previous deliveries of other types of nucleotides will have dissociated (e.g., to binary complex form) since the other types of nucleotides had been removed. As such, ternary complexes formed from each type of nucleotide can be identified based on the expectation that one type of ternary complex will be most prominent in each examination. For example, when ternary complex is detected based on recruitment of a labeled polymerase or labeled nucleotide to primed-template nucleic acids in an array, the array features having the highest signal can be identified as the features where ternary complex has formed. The type of ternary complex (e.g., the type of nucleotide present in the ternary complex) that forms at each of the features can be deduced from knowledge of which nucleotide was delivered prior to the examination step.

In this mode, the different types of ternary complexes need not be distinguished by unique labels. Rather, the different types of ternary complexes can be distinguished based on temporal information pertaining to when they formed and which nucleotide type was delivered to induce formation. If desired, the different types of ternary complexes can be distinguishably labeled. For example, each nucleotide type can have a label that produces a signal that is distinguished from all other nucleotide types used. Distinguishable labels can provide the advantage of increasing the speed of detection since a single examination step can be carried out after multiple different types of nucleotides have been delivered. Time savings can be achieved by simultaneously delivering two or more distinguishably labeled nucleotide types in a method set forth herein. If desired, examination can occur after each nucleotide delivery even when using distinguishable labels to identify different types of ternary complexes.

A method of the present disclosure can be performed in a mode whereby different nucleotide types are serially delivered to a vessel where ternary complex is to be formed and examined. In this mode, a first nucleotide type can be delivered to a reaction vessel and then a second nucleotide type can be delivered to the vessel such that the two nucleotide types accumulate in the vessel. When the vessel contains a variety of different primed-template nucleic acids, for example an array or other multiplex format, multiple different types of ternary complexes can accumulate in the vessel. Polymerase can be added initially to create conditions that facilitate ternary complex formation with the first nucleotide. New polymerase can be, but need not be, added in a subsequent delivery to facilitate ternary complex formation with a subsequently delivered nucleotide.

When performing the methods in a mode whereby different nucleotide types are serially delivered to a reaction vessel such that the different nucleotides accumulate, examination of the vessel for ternary complexes can be carried out after each delivery. In this mode, ternary complexes of different types (e.g., ternary complexes that differ in the type of nucleotide that is present) will form after each delivery. Ternary complexes that had formed in previous deliveries of other types of nucleotides will also be present in the vessel. As such, ternary complexes formed from each type of nucleotide can be identified based on the appearance of newly formed ternary complex from one examination to the next. For example, when ternary complex is detected based on recruitment of a labeled polymerase or labeled nucleotide to primed-template nucleic acids in an array, the array features having increased signal intensity compared to the signal intensity detected for that feature in previous examinations can be identified as the features where new ternary complex has formed. The type of ternary complex (e.g., the type of nucleotide present in the ternary complex) that forms at each of the features can be deduced from knowledge of which nucleotide was delivered prior to the examination step where new ternary complex signal arose.

Thus, the different types of ternary complexes need not be distinguished by unique labels. Rather, the different types of ternary complexes can be distinguished based on temporal information pertaining to when they formed and which nucleotide type was delivered to induce formation of the ternary complex. If desired, the different types of ternary complexes can be distinguishably labeled. For example, two or more nucleotide types can have labels that produce signals that are distinguished from each other. In some embodiments, all nucleotide types can be distinguished based on unique labels. Thus, labels can distinguish nucleotides that pair with one type of nucleotide in a template from nucleotides that pair with all other nucleotide types in the template. Distinguishable labels can provide the advantage of increasing the speed of detection since a single examination step can be carried out after all nucleotides have been serially delivered. If desired, examination can occur after each nucleotide delivery even when using distinguishable labels to identify different types of ternary complexes.

This disclosure further provides a method for identifying a nucleotide in a primed-template nucleic acid. The method can include the steps of (a) providing a vessel having a primed-template nucleic acid, polymerase and a nucleotide cognate of a first base type; (b) examining the vessel for a stabilized ternary complex including the polymerase and the nucleotide cognate of the first base type bound at a base position of the primed-template nucleic acid; (c) washing the vessel with an aqueous solution including a polyol, diol, sulfone or sulfoxide, thereby removing the nucleotides from the vessel and retaining the primed-template nucleic acid and the polymerase from step (b); (d) delivering a nucleotide cognate of a second base type to the vessel after step (c); (e) examining the vessel for a stabilized ternary complex including the polymerase and the nucleotide cognate of the second base type bound at the base position of the primed-template nucleic acid; and (f) identifying the type of nucleotide at the base position of the primed-template nucleic acid.

A method of this disclosure can include a step for detecting a ternary complex. Embodiments of the methods exploit the specificity with which a polymerase can form a stabilized ternary complex with a primed-template nucleic acid and a next correct nucleotide. The next correct nucleotide can be non-covalently bound to the stabilized ternary complex, interacting with the other members of the complex solely via non-covalent interactions. Useful methods and compositions for forming a stabilized ternary complex are set forth in further detail below and in U.S. Pat. App. Pub. Nos. 2017/0022553 A1 published on Jan. 26, 2017; 2018/0044727 A1 published on Feb. 15, 2018; 2018/0187245 A1 published on Jul. 5, 2018 and 2018/0208983 A1 published on Jul. 26, 2018, each of which is incorporated herein by reference.

Typically, examination is carried out separately and discretely from primer extension, for example, due to a reagent exchange or wash that intervenes examination and extension. Alternatively, examination and primer extension steps can occur in the same mixture in some embodiments.

Generally, detection can be achieved in an examination step by methods that perceive a property that is intrinsic to a ternary complex or a label moiety attached thereto. Exemplary properties upon which detection can be based include, but are not limited to, mass, electrical conductivity, energy absorbance, luminescence (e.g., fluorescence) or the like. Detection of luminescence can be carried out using methods known in the art pertaining to nucleic acid arrays. A luminophore can be detected based on any of a variety of luminescence properties including, for example, emission wavelength, excitation wavelength, fluorescence resonance energy transfer (FRET) intensity, quenching, anisotropy or lifetime. Other detection techniques that can be used in a method set forth herein include, for example, mass spectrometry which can be used to perceive mass; surface plasmon resonance which can be used to perceive binding at a surface; absorbance which can be used to perceive the wavelength of the energy a label absorbs; calorimetry which can be used to perceive changes in temperature due to presence of a label; electrical conductance or impedance which can be used to perceive electrical properties of a label, or other known analytic techniques. Examples of reagents and conditions that can be used to create, manipulate and detect stabilized ternary complexes include, for example, those set forth in U.S. Pat. App. Pub. Nos. 2017/0022553 A1 published on Jan. 26, 2017; 2018/0044727 A1 published on Feb. 15, 2018; 2018/0187245 A1 published on Jul. 5, 2018; and 2018/0208983 A1 published on Jul. 26, 2018, each of which is incorporated herein by reference.

Some embodiments of the methods set forth herein utilize two or more distinguishable signals to distinguish stabilized ternary complexes from each other and/or to distinguish one base type in a template nucleic acid from another base type. For example, two or more luminophores can be distinguished from each other based on unique optical properties such as unique wavelength for excitation or unique wavelength of emission. In some embodiments, a method can distinguish different stabilized ternary complexes based on differences in luminescence intensity. For example, a first ternary complex can be detected in a condition where it emits less intensity than a second ternary complex. Such intensity scaling (sometimes called ‘grey scaling’) can exploit distinguishable intensity differences. Exemplary differences include a particular stabilized ternary complex having an intensity that is at most 10%, 25%, 33%, 50%, 66%, or 75% compared to the intensity of another stabilized ternary complex that is to be detected.

Intensity differences can result from using different luminophores, for example, each having a different extinction coefficient (i.e., resulting in different excitation properties) and/or different luminescence quantum yield (i.e., resulting in different emission properties). Alternatively, the same luminophore type can be used but can be present in different amounts. For example, all members of a first population of ternary complexes can be labeled with a particular luminophore, whereas a second population has only half of its members labeled with the luminophore. In this example, the second population would be expected to produce half the signal of the first population. The second population can be produced, for example, by using a mixture of labeled nucleotides and unlabeled nucleotides (in contrast to the first population containing primarily labeled nucleotides). Similarly, the second population can be produced, for example, by using a mixture of labeled polymerases and unlabeled polymerases (in contrast to the first population containing primarily labeled polymerases). In an alternative labeling scheme, a first population of ternary complexes can include polymerase molecules that have multiple labels that produce a particular luminescent signal and a second population of ternary complexes can include polymerase molecules that each have only one of the labels that produces the luminescent signal.

A method for identifying a nucleotide in a primed-template nucleic acid can include the steps of (a) providing a vessel having a primed-template nucleic acid, polymerase and a nucleotide cognate of a first base type; (b) examining the vessel for a stabilized ternary complex including the polymerase and the nucleotide cognate of the first base type bound at a base position of the primed-template nucleic acid; (c) washing the vessel with an aqueous solution including a polyol, diol, sulfone, sulfoxide or a combination thereof, thereby removing the nucleotides from the vessel and retaining the primed-template nucleic acid and the polymerase from step (b); (d) delivering a nucleotide cognate of a second base type to the vessel after step (c); (e) examining the vessel for a stabilized ternary complex including the polymerase and the nucleotide cognate of the second base type bound at the base position of the primed-template nucleic acid; and (f) identifying the type of nucleotide at the base position of the primed-template nucleic acid, wherein the vessel of step (a) further includes a nucleotide cognate of a third base type, and wherein step (b) includes examining the vessel for a stabilized ternary complex having the polymerase and (i) the nucleotide cognate of the first base type bound at the base position of the primed-template nucleic acid or (ii) the nucleotide cognate of the third base type bound at the base position of the primed-template nucleic acid.

Optionally the above method can be configured such that the nucleotide cognate of the first base type has an exogenous label and the nucleotide cognate of the third base type has an exogenous label. The exogenous label of the nucleotide cognate of the first base type can be different from the exogenous label of the nucleotide cognate of the third base type. In this configuration, step (b) can be carried out to distinguish signals from the different exogenous labels. As a further option, step (c) can further include delivering a nucleotide cognate of a fourth base type to the vessel, and step (d) can include examining the vessel for a stabilized ternary complex having the polymerase and (i) the nucleotide cognate of the second base type bound at the base position of the primed-template nucleic acid or (ii) the nucleotide cognate of the fourth base type bound at the base position of the primed-template nucleic acid. The second base type can have an exogenous label and the nucleotide cognate of the fourth base type can have an exogenous label. The exogenous label on the nucleotide cognate of the second base type can be different from the exogenous label on the nucleotide cognate of the fourth base type. In this configuration, step (b) can further include distinguishing signals from the different exogenous labels.

Alternatively or additionally, the exogenous label on the nucleotide cognate of the first base type can produce the same signal as the exogenous label on the nucleotide cognate of the third base type. Alternatively or additionally, the exogenous label on the nucleotide cognate of the second base type can produce a signal that is not distinguished from the signal produced by the exogenous label on the nucleotide cognate of the fourth base type.

In some embodiments, the examination step is carried out in a way that the identity of at least one nucleotide type is imputed, for example, as set forth in U.S. Pat. Nos. 9,951,385 and 10,161,003, each of which is incorporated herein by reference. Alternatively or additionally to using imputation, an examination step can use disambiguation to identify one or more nucleotide types, for example, as set forth in U.S. Pat. Nos. 9,951,385 and 10,161,003, each of which is incorporated herein by reference.

A method for identifying a nucleotide in a primed-template nucleic acid can be performed in a mode whereby different nucleotide types are serially delivered and then removed from a vessel where ternary complex is to be formed and examined. Accordingly, the method can include the steps of (a) providing a vessel having a primed-template nucleic acid, polymerase and a nucleotide cognate of a first base type; (b) examining the vessel for a stabilized ternary complex including the polymerase and the nucleotide cognate of the first base type bound at a base position of the primed-template nucleic acid; (c) washing the vessel with an aqueous solution including a polyol, diol, sulfone or sulfoxide, thereby removing the nucleotides from the vessel and retaining the primed-template nucleic acid and the polymerase from step (b); (d) delivering a nucleotide cognate of a second base type to the vessel after step (c); (e) examining the vessel for a stabilized ternary complex including the polymerase and the nucleotide cognate of the second base type bound at the base position of the primed-template nucleic acid; and (f) identifying the type of nucleotide at the base position of the primed-template nucleic acid.

In some embodiments, a method for identifying a nucleotide in a primed-template nucleic acid can include the steps of (a) providing a vessel having a primed-template nucleic acid, polymerase and a nucleotide cognate of a first base type; (b) examining the vessel for a stabilized ternary complex including the polymerase and the nucleotide cognate of the first base type bound at a base position of the primed-template nucleic acid; (c) washing the vessel with an aqueous solution including a polyol, diol, sulfone or sulfoxide, thereby removing the nucleotides from the vessel and retaining the primed-template nucleic acid and the polymerase from step (b); (d) delivering a nucleotide cognate of a second base type to the vessel after step (c); (e) examining the vessel for a stabilized ternary complex including the polymerase and the nucleotide cognate of the second base type bound at the base position of the primed-template nucleic acid; (f) identifying the type of nucleotide at the base position of the primed-template nucleic acid; (g) delivering a nucleotide cognate of a third base type to the vessel, whereby the vessel retains the primed-template nucleic acid and the polymerase from step (b); and (h) examining the vessel for a stabilized ternary complex having the polymerase and the nucleotide cognate of the third base type bound at the base position of the primed-template nucleic acid.

In some embodiments, a method for identifying a nucleotide in a primed-template nucleic acid can include the steps of (a) providing a vessel having a primed-template nucleic acid, polymerase and a nucleotide cognate of a first base type; (b) examining the vessel for a stabilized ternary complex including the polymerase and the nucleotide cognate of the first base type bound at a base position of the primed-template nucleic acid; (c) washing the vessel with an aqueous solution including a polyol, diol, sulfone or sulfoxide, thereby removing the nucleotides from the vessel and retaining the primed-template nucleic acid and the polymerase from step (b); (d) delivering a nucleotide cognate of a second base type to the vessel after step (c); (e) examining the vessel for a stabilized ternary complex including the polymerase and the nucleotide cognate of the second base type bound at the base position of the primed-template nucleic acid; (f) identifying the type of nucleotide at the base position of the primed-template nucleic acid; (g) delivering a nucleotide cognate of a third base type to the vessel, whereby the vessel retains the primed-template nucleic acid and the polymerase from step (b); (h) examining the vessel for a stabilized ternary complex having the polymerase and the nucleotide cognate of the third base type bound at the base position of the primed-template nucleic acid; (i) delivering a nucleotide cognate of a fourth base type to the vessel, whereby the vessel retains the primed-template nucleic acid and the polymerase from step (b); and (j) examining the vessel for a stabilized ternary complex including the polymerase and the nucleotide cognate of the fourth base type bound at the base position of the primed-template nucleic acid.

A method of the present disclosure can include a step of modifying a primer, for example, to extend the primer by addition of one or more nucleotides. In some embodiments, a nucleotide that is added to a primer will include a reversible terminator moiety. The reversible terminator moiety can provide the non-limiting benefits of preventing more than one nucleotide from being added to the primer during the extension process and stabilizing ternary complex formation at the 3′ end of the primer during an examination process.

Typically, a nucleotide, such as a reversibly terminated nucleotide, that is added to a primer in a method set forth herein does not have an exogenous label. This is because the extended primer need not be detected in a method set forth herein. However, if desired, one or more types of reversibly terminated nucleotides used in a method set forth herein can be detected, for example, via exogenous labels attached to the nucleotides.

A primer extension process or a process of forming a ternary complex need not use a labeled polymerase. For example, a polymerase that is used for an extension step need not be attached to an exogenous label (e.g., covalently or otherwise). Alternatively, a polymerase that is used for primer extension can include an exogenous label, for example, a label that was used in a previous or subsequent examination step.

Examples of reagents and conditions that can be used for a polymerase-based primer extension step include, for example, those set forth in U.S. Pat. App. Pub. Nos. 2017/0022553 A1 published on Jan. 26, 2017; 2018/0044727 A1 published on Feb. 15, 2018; and 2018/0187245 A1 published on Jul. 5, 2018, each of which is incorporated herein by reference. Exemplary reversible terminator moieties, methods for incorporating them into primers and methods for modifying the primers for further extension (often referred to as ‘deblocking’) are set forth in U.S. Pat. Nos. 7,544,794; 7,956,171; 8,034,923; 8,071,755; 8,808,989; and 9,399,798. Further examples are set forth in Bentley et al., Nature 456:53-59 (2008), WO 04/018497 published on Mar. 4, 2004; U.S. Pat. No. 7,057,026; WO 91/06678 published on May 16, 1991; WO 07/123744 published on Nov. 11, 2007; U.S. Pat. Nos. 7,329,492; 7,211,414; 7,315,019; 7,405,281, and US Pat. App. Pub. No. 2008/0108082 A1 published on May 8, 2008, each of which is incorporated herein by reference.

Accordingly, a method for identifying a nucleotide in a primed-template nucleic acid can include the steps of (a) providing a vessel having a primed-template nucleic acid, polymerase and a nucleotide cognate of a first base type; (b) examining the vessel for a stabilized ternary complex including the polymerase and the nucleotide cognate of the first base type bound at a base position of the primed-template nucleic acid; (c) washing the vessel with an aqueous solution including a polyol, diol, sulfone or sulfoxide, thereby removing the nucleotides from the vessel and retaining the primed-template nucleic acid and the polymerase from step (b); (d) delivering a nucleotide cognate of a second base type to the vessel after step (c); (e) examining the vessel for a stabilized ternary complex including the polymerase and the nucleotide cognate of the second base type bound at the base position of the primed-template nucleic acid; (f) identifying the type of nucleotide at the base position of the primed-template nucleic acid; (g) adding a nucleotide to the primer of the primed-template nucleic acid, whereby the vessel includes an extended primed-template nucleic acid; (h) delivering a second polymerase and a nucleotide cognate of the first base type to the vessel; and (i) repeating steps (b) through (f) using the extended primed-template instead of the primed-template nucleic acid and using the second polymerase instead of the polymerase.

Some embodiments of the methods set forth herein can employ a reversibly terminated primer and/or reversibly terminated nucleotides. The reversible terminator moiety on these species can be removed or modified to extendable form via a deblocking process. A deblocking process when included in a method set forth herein can facilitate sequencing of a primed-template nucleic acid. The deblocking process can be used to convert a reversibly terminated primer into an extendable primer. Primer extension can then be used to move the site of ternary complex formation to a different location along the template nucleic acid. Repeated cycles of extension, examination and deblocking can be used to reveal the sequence of a template nucleic acid. Each cycle reveals a subsequent base in the template nucleic acid. Sequencing techniques that step along a template by blocking and deblocking a primer are referred to as using cyclic reversible termination (CRT). Such CRT techniques can be used, for example, in Sequencing By Binding™, Sequencing By Synthesis, Sequencing By Ligation or Pyrosequencing™ sequencing methods. Exemplary reversible terminator moieties, methods for incorporating them into primers and methods for modifying the primers for further extension (often referred to as ‘deblocking’) are set forth in U.S. Pat. Nos. 7,427,673; 7,414,116; 7,544,794; 7,956,171; 8,034,923; 8,071,755; 8,808,989; and 9,399,798. Further examples are set forth in Bentley et al., Nature 456:53-59 (2008), WO 04/018497 published on Mar. 4, 2004; U.S. Pat. No. 7,057,026; WO 91/06678 published on May 16, 1991; WO 07/123744 published on Nov. 1, 2007; U.S. Pat. Nos. 7,329,492; 7,211,414; 7,315,019; 7,405,281, and US 2008/0108082 published on May 8, 2008, each of which is incorporated herein by reference.

A sequencing method can include multiple repetitions of cycles, or steps within cycles, set forth herein. For example, a cycle that includes examination and primer extension steps can be repeated multiple times to detect nucleotide positions along a template nucleic acid. Optionally, the cycle can further include steps of deblocking primers, or washing away unused reactants or spent products between various steps. Accordingly, a primed-template nucleic acid can be subjected at least 2, 5, 10, 25, 50, 100, 150, 200, or more repeated cycles of a method set forth herein. Fewer cycles can be carried out when shorter read lengths are adequate. As such, a primed-template nucleic acid can be subjected to at most 200, 150, 100, 50, 25, 10, 5, or 2 cycles of a method set forth herein.

In some embodiments, a sequencing method can be carried out for a predetermined number of repeated cycles. Alternatively, the cycles can be repeated until a particular empirically observed state is reached. For example, cycles can be repeated so long as signal is above an observable threshold, noise is below an observable threshold or signal-to-noise ratio is above an observable threshold.

Although embodiments of the present disclosure are exemplified herein with regard to sequencing reactions that employ repeated cycles, the cycles need not be repeated nor do the cycles need to include primer extension steps. For example, genotyping can be carried out by examining a single nucleotide position in a template nucleic acid via formation of a stabilized ternary complex. Genotyping can be carried out using serial delivery and/or accumulation of nucleotide cognates for different base types. Examples of genotyping techniques that can be modified to employ the nucleotide delivery methods set forth herein include those set forth in U.S. Pat. No. 9,932,631, which is incorporated herein by reference.

Disclosed herein include a composition containing a plurality of polymerase-nucleic acid complexes in contact with an aqueous solution, wherein each of the plurality of polymerase-nucleic acid complex comprises a polymerase and the nucleic acid comprises a primed-template nucleic acid in contact with an aqueous solution, wherein the aqueous solution comprises a polyol, alcohol, aliphatic diol, sulfone, or sulfoxide, or a combination thereof. In the composition, at least multiple (e.g., a subset) of the polymerase-nucleic acid complexes can be ternary complexes further comprising nucleotides, for example at least 20%, 25%, 30%, 35%, 40%, 45%, or 50% of the polymerase-nucleic acid complexes can be ternary complexes further comprising nucleotides. For example, 25%, 30%, 35%, 40%, 45%, 50%, 60%, 70%, 80%, 90%, 95%, 99%, or a number or a range between any two of these values, of the polymerase-nucleic acid complexes are ternary complexes further comprising nucleotides. In some embodiments, at least multiple (e.g., a subset) of the polymerase-nucleic acid complexes do not comprise nucleotides, for example at most 50%, 45%, 40%, 35%, 30%, 25%, 20%, 15%, 10%, or 5% of the polymerase-nucleic acid complexes do not comprise nucleotides. For example, 5%, 10%, 15%, 20%, 25%, 30%, 35%, 40%, 45%, 50%, or a number or a range between any two of these values, of the polymerase-nucleic acid complexes do not comprise nucleotides. The aqueous solution can further comprise Lithium, Betaine, or both.

The present disclosure further provides a system for detecting an analyte set forth herein, such as a ternary complex or component that is capable of forming a ternary complex. A system of the present disclosure can be configured to perform one or more of the methods set forth herein. For example, a system can be configured to produce and detect ternary complexes formed between a polymerase and a primed-template nucleic acid in the presence of nucleotides to identify one or more bases in a template nucleic acid sequence. Optionally, the system includes components and reagents for performing one or more steps set forth herein including, but not limited to, forming at least one stabilized ternary complex between a primed-template nucleic acid, polymerase and next correct nucleotide; detecting the stabilized ternary complex(es); extending the primer of primer-template hybrid(s); deblocking a reversibly terminated primer(s); and/or identifying a nucleotide, or sequence of nucleotides in one or more templates.

A system of the present disclosure can include a vessel, solid support or other apparatus for carrying out a nucleic acid detection method. For example, the system can include an array, flow cell, multi-well plate, test tube, channel in a substrate, collection of droplets or vesicles, tray, centrifuge tube, tubing or other convenient apparatus. The apparatus can be removable, thereby allowing it to be placed into or removed from the system. As such, a system can be configured to process a plurality of apparatus (e.g., vessels or solid supports) sequentially or in parallel. The system can include a fluidic component having reservoirs for containing one or more of the reagents set forth herein (e.g., polymerase, primer, template nucleic acid, nucleotide(s) for ternary complex formation, nucleotides for primer extension, deblocking reagents, ternary complex inhibitors, or mixtures of such components). The fluidic system can be configured to deliver reagents to a vessel or solid support, for example, via channels or droplet transfer apparatus (e.g., electrowetting apparatus). Any of a variety of detection apparatus can be configured to detect the vessel or solid support where reagents interact. Examples include luminescence detectors, surface plasmon resonance detectors and others known in the art. Exemplary systems having fluidic and detection components that can be readily modified for use in a system herein include, but are not limited to, those set forth in US Pat. App. Pub. No. 2018/0280975A1 published on Oct. 4, 2018; U.S. Pat. Nos. 8,241,573; 7,329,860 and 8,039,817; or US Pat. App. Pub. Nos. 2009/0272914 A1

published on Nov. 5, 2009 and 2012/0270305 A1 published on Oct. 25, 2012, each of which is incorporated herein by reference.

Optionally, a system of the present disclosure further includes a computer processing unit (CPU) that is configured to operate system components. The same or different CPU can interact with the system to acquire, store and process signals (e.g., signals detected in a method set forth herein). In some embodiments, a CPU can be used to determine, from the signals, the identity of the nucleotide that is present at a particular location in a template nucleic acid. In some cases, the CPU will identify a sequence of nucleotides for the template from the signals that are detected.

A useful CPU can include one or more of a personal computer system, server computer system, thin client, thick client, hand-held or laptop device, multiprocessor system, microprocessor-based system, set top box, programmable consumer electronic, network PC, minicomputer system, mainframe computer system, smart phone, and distributed cloud computing environments that include any of the above systems or devices, and the like. The CPU can include one or more processors or processing units, a memory architecture that may include RAM and non-volatile memory. The memory architecture may further include removable/non-removable, volatile/non-volatile computer system storage media. Further, the memory architecture may include one or more readers for reading from and writing to a non-removable, non-volatile magnetic media, such as a hard drive, a magnetic disk drive for reading from and writing to a removable, non-volatile magnetic disk, and/or an optical disk drive for reading from or writing to a removable, non-volatile optical disk such as a CD-ROM or DVD-ROM. The CPU may also include a variety of computer system readable media. Such media may be any available media that is accessible by a cloud computing environment, such as volatile and non-volatile media, and removable and non-removable media.

The memory architecture may include at least one program product having at least one program module implemented as executable instructions that are configured to carry out one or more steps of a method set forth herein. For example, executable instructions may include an operating system, one or more application programs, other program modules, and program data. Generally, program modules may include routines, programs, objects, components, logic, data structures, and so on, that perform particular tasks set forth herein.

The components of a CPU may be coupled by an internal bus that may be implemented as one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

A CPU can optionally communicate with one or more external devices such as a keyboard, a pointing device (e.g., a mouse), a display, such as a graphical user interface (GUI), or other device that facilitates interaction with the nucleic acid detection system. Similarly, the CPU can communicate with other devices (e.g., via network card, Bluetooth™, WiFi, modem, etc.). Such communication can occur via I/O interfaces. Still yet, a CPU of a system herein may communicate with one or more networks such as a local area network (LAN), a general wide area network (WAN), and/or a public network (e.g., the Internet) via a suitable network adapter.

### EXAMPLE I

### Evaluating Compounds for Stripping Nucleotides from Ternary Complexes

This example demonstrates the effect of various compounds on stripping nucleotides from polymerase-nucleic acid complexes.

### Materials and Methods

Sequencing was carried out as set forth in U.S. patent application Ser. No. 16/700,422 published as US Pat. App. Pub. No. 20200171498 on Jun. 4, 2020 (which is incorporated herein by reference) with certain details and modifications as set forth in the following description.

Template nucleic acid strands synthesized in 12 PCR reactions were prepared, and then independently bound to beads. This resulted in a population of 12 bead types, where each bead harbored a homogenous collection of one of the 12 template strands. Beads harboring immobilized template strands were then attached to the inner surface of a flow cell. Sequencing was performed for 22 cycles, where each cycle included subroutines for (i) Extension: adding a reversibly terminated nucleotide to primers that were hybridized to the immobilized templates, (ii) Examination: forming and detecting stabilized ternary complexes on the reversibly terminated, immobilized primer-template hybrids, and (iii) Activation: cleaving the reversible terminator from the extended primers.

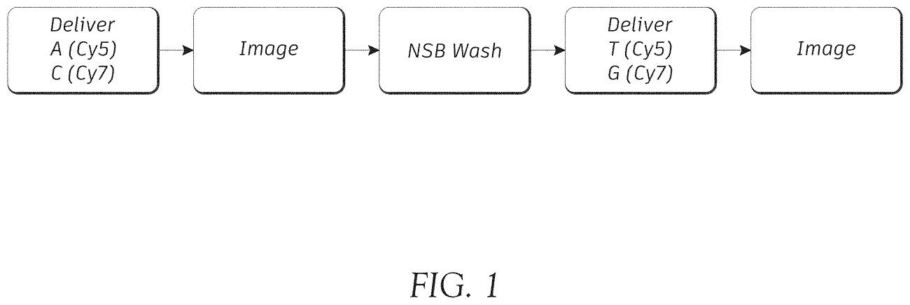

The extension and Activation subroutines were carried out essentially as set forth in Example 1 of U.S. patent application Ser. No. 16/700,422. The Examination subroutine was performed in a 2×2 format as diagrammed in FIG. 1. Specifically, a mixture of Cy5 labelled dATP, Cy7 labelled dCTP, unlabeled dTTP and unlabeled dGTP was delivered to the flow cell that contained polymerase-nucleic acid complexes on the beads, the system paused fluid flow to allow nucleotides to bind at cognate positions of the templates, thereby forming ternary complexes. Free nucleotide was removed from the flow cell by flowing IMG reagent. The IMG reagent included LiCl, betaine, Tween-80, KCl, Ammonium Sulfate, hydroxylamine, and EDTA which stabilized the ternary complexes after removal of free nucleotides (see U.S. Pat. No. 10,400,272, which is incorporated herein by reference). The flow cell was then imaged via fluorescence microscopy to detect ternary complexes that contained a labeled nucleotide that was a cognate for the next correct nucleotide in each of the template nucleic acids. The flow cell was then washed with NSB solutions having various compositions as set forth below. After the NSB wash, a mixture of Cy5 labelled dTTP, Cy7 labelled dGTP, unlabeled dCTP and unlabeled dATP was delivered to the flow cell, and the system paused fluid flow to allow nucleotides to bind, thereby forming ternary complexes. Free nucleotide was removed by flowing IMG reagent and then the flow cell was again imaged.

The NSB solutions consisted of 50 mM Tricine, 43.75 mM KCl, 6.25 mM KOH, 5 mM O-tertbutylhydroxylamine hydrochloride, 100 μM EDTA, 0.05% (v/v) ProClin 950 (Sigma, Cat. 46878-U), 0.1% Tween-80, and one of the candidate stripping agents shown in Table 1.

TABLE 1

| Candidate Compound | Concentration in NSB | |
|---|---|---|
| Isopropanol (SOP) | 20% | v/v |
| 1,6 Hexanediol | 28% | w/v |
| 1,7 Heptanediol | 10% | v/v |
| 1,5 Pentanediol | 35% | w/v |
| Propylene Glycol | 35% | v/v |
| Hexylene Glycol | 20% | v/v |
| Sulfolane | 25% | v/v |
| DMSO | 26% | v/v |
| Ethyl Methyl Sulfone | 2.8% | w/v |
| PEG-35K | 8% | v/v |
| PVA | 3.2% | v/v |
| 2-methoxy ethanol | 21% | v/v |

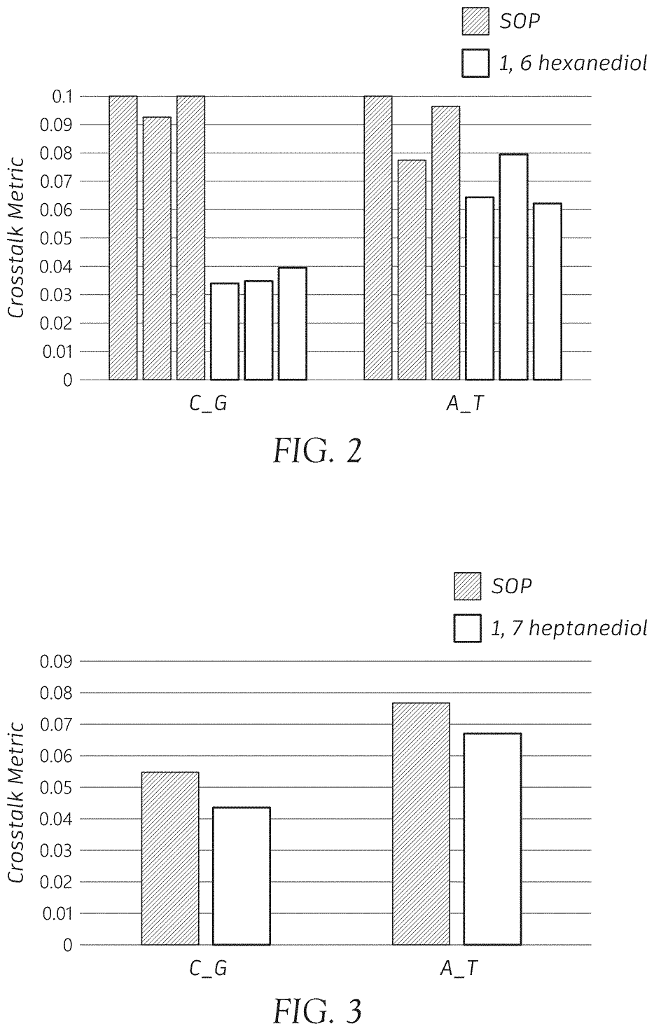

The second column of Table 1 lists the concentration of the respective candidate compound that yielded the results shown in FIGS. 2-9. The concentrations are provided as a percentage in weight of compound per total volume of NSB (w/v) or volume of compound per total volume of NSB (v/v). The concentration can also be expressed as a molar concentration and this can be helpful, for example, as a way to compare the amounts of each compound tested. For example, the molar concentration of 1,6 hexanediol and 1,7 pentanediol in NSB was lower than the molar concentration of isopropanol in the SOP NSB. The molar concentrations of 1,5 pentanediol, propylene glycol, hexylene glycol, sulfolane, DMSO, ethyl methyl sulfone and 2-mthoxy ethanol in NSB were similar to the molar concentration of isopropanol in the SOP NSB.

The effectiveness of each candidate compound was determined from the following crosstalk metrics. The A_T crosstalk metric indicates how much Cy5-dATP is carried over from the first exam such that it is detected in the second exam. Similarly, the C_G crosstalk metric indicates how much Cy7-dCTP is carried over from the first exam such that it is detected in the second exam. A lower value for the crosstalk metric indicates less carryover and is correlated with improved sequencing results. On the other hand, a higher value for the crosstalk metric indicates more carryover and lower quality sequencing results.

### Results

Results were evaluated as a comparison of crosstalk metrics measured for NSB having 20% v/v/isopropanol (SOP) compared to the crosstalk metric measured for NSB having each of the respective candidate compounds. In all cases, the comparisons were based on data obtained for sequencing runs performed in a single lane of a flow cell. The format of the run was as follows: Cycle_1(SOP)=>Cycle_2(candidate)=>Cycle_3(SOP)=>Cycle_4(candidate) and so on. The crosstalk metric was determined separately for the SOP and candidate cycles, respectively. This format was used to minimize the confounding influence of run-to-run variations when comparing between SOP and each candidate.

### Monohydroxy Alcohols

Alcohols such as ethanol, methanol, and isopropanol are useful compounds for stripping nucleotides from ternary complexes without disassociating the polymerase from the primed-template nucleic acid. Useful Alcohols include those that are water soluble (e.g., to at least 50% v/v), those having only a single hydroxyl, those that are primary alcohols, those that are aliphatic, and/or those having a molecular weight lower than about 100 g/mol. Non-limiting examples of alcohols are described in US Pat. App. Pub. No. 2020/0032317 A1 published on Jan. 30, 2020, which is incorporated herein by reference.

### Diols

The results for 1,6 hexanediol are shown in FIG. 2. The three separate bars for SOP and 1,6 hexanediol, respectively, represent results from separate cycles of the same sequencing run. The C_G data, which shows the crosstalk due to carryover of C signal from the first exam into the G channel for the second exam, was significantly lower for 1,6 hexanediol-based NSB. The A_T crosstalk is also modestly lower for 1,6 hexanediol-based NSB. Thus, NSB having 1,6 Hexanediol outperformed NSB having isopropanol even at lower molar concentrations than isopropanol. A further advantage of 1,6 Hexanediol is that it is non-flammable, so it does not need special storage like that for isopropanol.

The results for 1,7 heptanediol are shown in FIG. 3. The C_G data, which shows the crosstalk due to carryover of C signal from the first exam into the G channel for the second exam, was better for 1,7 heptanediol-based NSB. The A_T crosstalk was also better for 1,7 heptanediol-based NSB. Thus, NSB having 1,6 heptanediol outperformed NSB having isopropanol even at lower molar concentrations than isopropanol.

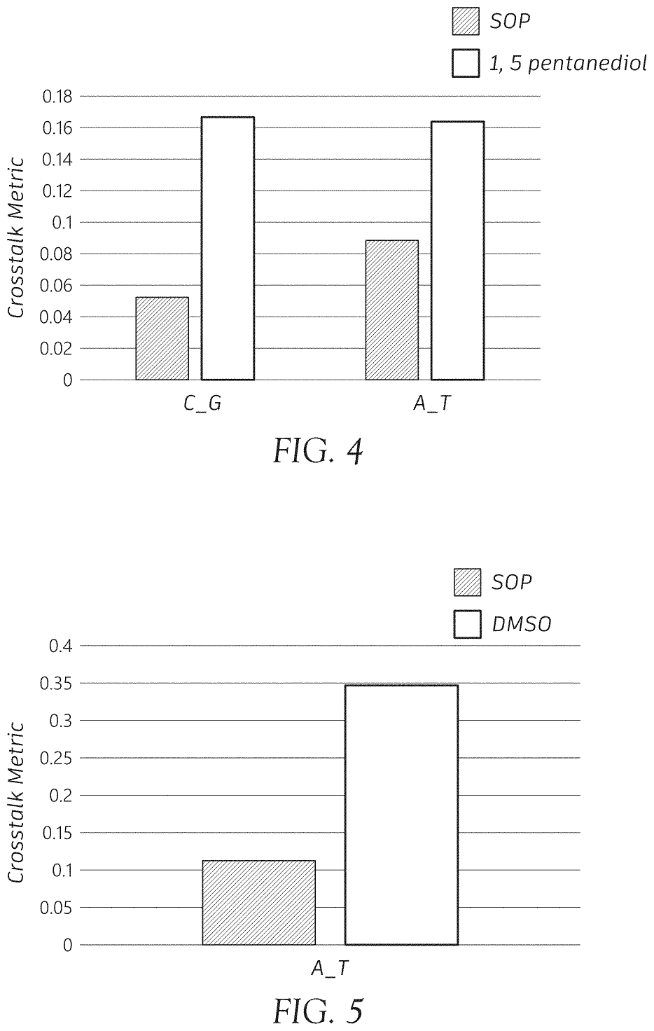

The results for 1,5 pentanediol are shown in FIG. 4. The C_G crosstalk and A_T crosstalk were stronger for NSB having 1,5 pentanediol compared to NSB having isopropanol. However, NSB having 1,5 pentanediol was still capable of maintaining relatively low crosstalk metrics of about 0.16 for both C_G crosstalk and A_T crosstalk.

Propylene glycol, 1,3 butanediol and hexylene glycol were also tested and, although capable of stripping nucleotides from ternary complexes while retaining polymerase association with primed-template nucleic acid, did not perform as well as isopropanol in NSB. 1,8 octanediol was not immediately soluble in NSB and was thus not tested in the sequencer.

### Sulfoxides and Sulfones

The results for dimethyl sulfoxide (DMSO) are shown in FIG. 5. The A_T crosstalk was stronger for NSB having DMSO compared to NSB having isopropanol. However, NSB having DMSO was still capable of maintaining a crosstalk metric below about 0.35 for A_T crosstalk (G_C crosstalk data was not available).

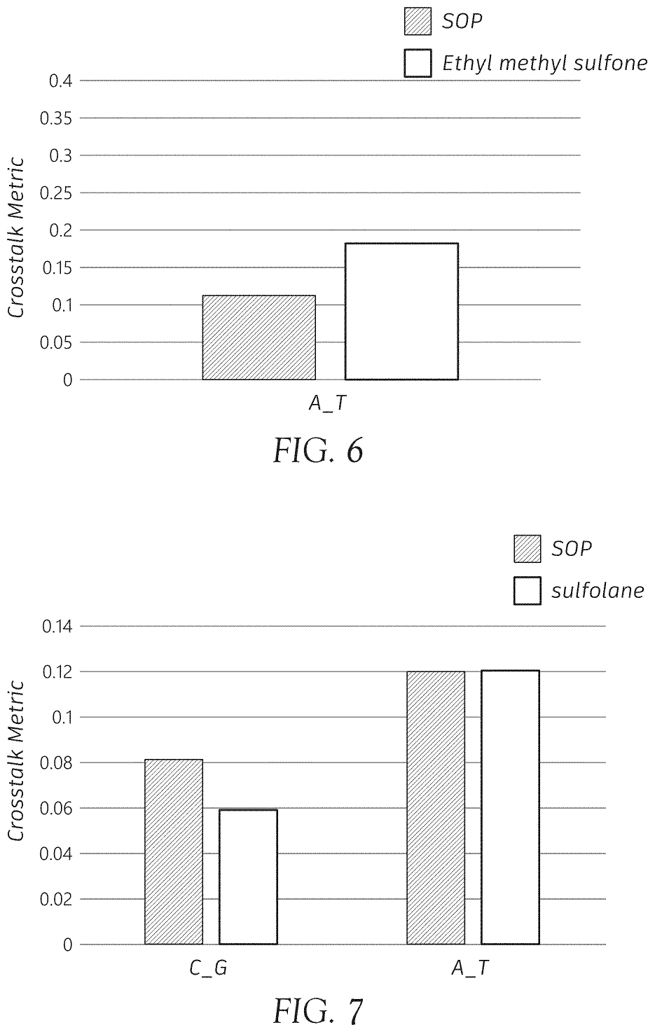

FIG. 6 shows results for ethyl methyl sulfone. The A_T crosstalk was stronger for NSB having ethyl methyl sulfone compared to NSB having isopropanol. However, NSB having ethyl methyl sulfone was still capable of maintaining a crosstalk metric below about 0.2 for A_T crosstalk (G_C crosstalk data was not available).

The results for sulfolane are shown in FIG. 7. The C_G data, which shows the crosstalk due to carryover of C signal from the first exam into the G channel for the second exam, was better for sulfolane-based NSB. The A_T crosstalk was comparable for sulfolane-based NSB and isopropanol-based NSB. Thus, NSB having sulfolane outperformed NSB having isopropanol.

### Polyols

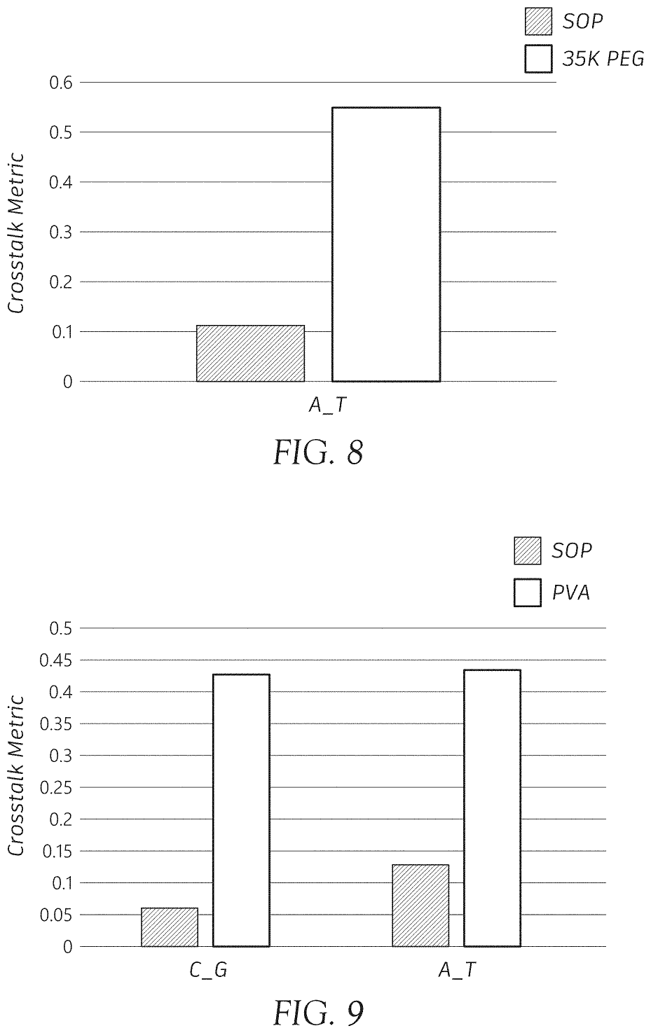

The results for polyethylene glycol (PEG) are shown in FIG. 8. The A_T crosstalk was stronger for NSB having PEG compared to NSB having isopropanol. However, NSB having PEG was still capable of maintaining a crosstalk metric below about 0.6 for A_T crosstalk (G C crosstalk data was not available). A 35K PEG polymer was used for these analyses. It is contemplated that other size polymers can be useful so long as they are soluble in aqueous solution.

FIG. 9 shows results for polyvinyl alcohol (PVA). The C_G crosstalk and A_T crosstalk were stronger for NSB having PVA compared to NSB having isopropanol. However, NSB having PVA was still capable of maintaining a crosstalk metric below about 0.45 for A_T crosstalk and G_C crosstalk.

### Aliphatic Compounds Having Both Hydroxyl Moieties and Another Heteroatom Moiety

Further tests indicated that 2-methoxy ethanol, tetraethylene glycol, 3-Amino-1-propanol can also be useful for stripping nucleotides from a polymerase-nucleic acid complex without dissociating the polymerase from the nucleic acid. Accordingly, the results demonstrated that alkoxy alcohols, ether alcohols and amino alcohols can be useful compounds.

### Other Concentrations Tested

In addition to the formulations listed in Table 1, other concentrations of several candidate compounds were also tested. Isopropanol when present in a range of 16-20% was effective, 1,6 hexanediol when present in a range of 10-31% was effective, 1.5 pentanediol when present in a range of 25-35% was effective, sulfolane when present in a range of 25-35% was effective, DMSO when present in a range of 21-26% was effective, and ethyl methyl sulfone when present in a range of 1.4-2.8% was effective. It is contemplated that other concentrations such as those set forth elsewhere herein can also be useful.

### EXAMPLE II

### Evaluating Stability of Foam in the Presence of Isopropanol and 1,6 Hexanediol

This example demonstrates that an aqueous solution having 1,6 hexanediol improves the stability of a fluid foam compared to an aqueous solution having isopropanol.

NSB fluid foam having 20% isopropanol (SOP) or 28% 1,6 hexanediol was flowed through a flow cell (40 μl/s flow rate, 300 μl volume, 35 psi positive pressure, 55° C.). The flow was paused and a first image of the foam was acquired immediately. The foam was allowed to sit for 60 seconds and then a second image was taken.

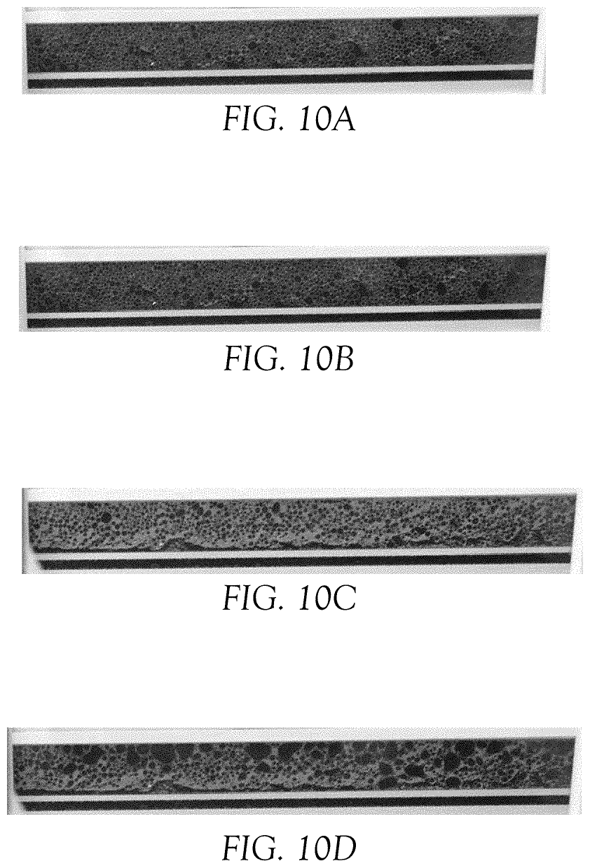

FIG. 10A shows the first image acquired for NSB fluid foam having 28% 1,6 hexanediol; FIG. 10B shows the second image acquired for NSB fluid foam having 28% 1,6 hexanediol; FIG. 10C shows the first image acquired for NSB fluid foam having 20% isopropanol (SOP); and FIG. 10D shows the second image acquired for NSB fluid foam having 20% isopropanol (SOP). The results indicated that although foam can be maintained to some degree in isopropanol, greater foam stability was observed in 1,6 hexanediol both before and after the 1 minute pause in flow.

Throughout this application various publications, patents and/or patent applications have been referenced. The disclosures of these documents in their entireties are hereby incorporated by reference in this application.

A number of embodiments have been described. Nevertheless, it will be understood that various modifications may be made. Accordingly, other embodiments are within the scope of the following claims.

What is claimed is:

1. A method for modifying polymerase-nucleic acid complexes, comprising:
(a) providing a plurality of polymerase-nucleic acid complexes each comprises a polymerase and a primed-template nucleic acid, wherein at least a subset of the polymerase-nucleic acid complexes are ternary complexes further comprising nucleotides; and
(b) contacting the plurality of polymerase-nucleic acid complexes with an aqueous solution comprising a polyol, alcohol, aliphatic diol, sulfone, sulfoxide, or a combination thereof, thereby dissociating the nucleotides from the subset of polymerase-nucleic acid complexes.

2. The method of claim 1, wherein the aqueous solution further comprises Lithium, Betaine, or both.

3. The method of claim 1 or 2, wherein the plurality of polymerase-nucleic acid complexes are immobilized on a surface.

4. The method of any one of claims 1-3, wherein the plurality of polymerase-nucleic acid complexes are present in a vessel.

5. The method of claim 4, wherein contacting the plurality of polymerase-nucleic acid complexes with the aqueous solution comprises washing the surface on which the plurality polymerase-nucleic acid complexes are immobilized with the aqueous solution, thereby removing the nucleotides from the subset of polymerase-nucleic acid complexes in the vessel.

6. The method of any one of claims 1-5, wherein after contacting the plurality of polymerase-nucleic acid complexes with the aqueous solution, at least 50% of the polymerases are retained in the plurality of polymerase-nucleic acid complexes.

7. The method of any one of claims 1-5, wherein after contacting the plurality of polymerase-nucleic acid complexes with the aqueous solution, at least 70% of the polymerases are retained in the plurality of polymerase-nucleic acid complexes.

8. The method of any one of claims 1-5, wherein after contacting the plurality of polymerase-nucleic acid complexes with the aqueous solution, at most 5% of the polymerases dissociate from the plurality of polymerase-nucleic acid complexes.

9. The method of any one of claims 1-5, wherein after contacting the plurality of polymerase-nucleic acid complexes with an aqueous solution, at most 20% of the polymerases dissociate from the plurality of polymerase-nucleic acid complexes.

10. The method of any one of claims 1-9, wherein dissociating the nucleotides from the subset of polymerases-nucleic acid complexes comprises dissociating at least 50% of the nucleotides from the ternary complexes.

11. The method of any one of claims 1-9, wherein dissociating the nucleotides from the subset of polymerases-nucleic acid complexes comprises dissociating at least 70% of the nucleotides from the ternary complexes.

12. The method of any one of claims 4-11, wherein step (a) comprises contacting a plurality of polymerases with a plurality of primed-template nucleic acids and with a plurality of nucleotides, thereby providing a plurality of surface-immobilized polymerase-nucleic acid complexes in the vessel, each of the surface-immobilized polymerase-nucleic acid complexes comprising a polymerase of the plurality of polymerases and a primed-template nucleic acid of the plurality of primed-template nucleic acids, the nucleotides in the ternary complexes comprising nucleotides of the plurality of nucleotides.

13. The method of claim 12, wherein the plurality of polymerases is attached to the surface prior to the contacting with the plurality of primed-template nucleic acids.

14. The method of claim 12, wherein the plurality of primed-template nucleic acids is attached to the surface prior to the contacting with the plurality of polymerases.

15. The method of any one of claims 12-14, wherein the plurality of polymerases is simultaneously contacted with the plurality of primed-template nucleic acids and with the plurality of nucleotides.

16. The method of any one of claims 12-14, wherein the plurality of primed-template nucleic acids is sequentially contacted with the plurality of polymerases and then with the plurality of nucleotides.

17. The method of any one of claims 12-14, wherein the plurality of polymerases is sequentially contacted with the plurality of nucleotides and then with the plurality of primed-template nucleic acids.

18. The method of any one of claims 12-17, wherein the plurality of nucleotides comprises cognates for at least two different types of bases.

19. The method of any one of claims 12-17, wherein the plurality of nucleotides comprises cognates for at least four different types of bases.

20. The method of any one of claims 3-19, wherein the primed-template nucleic acids of the surface-immobilized polymerase-nucleic acid complexes are covalently attached to the surface.

21. The method of any one of claims 3-20, wherein the polymerases of the surface-immobilized polymerase-nucleic acid complexes are covalently attached to the surface.

22. The method of any one of claims 3-21, wherein the nucleotides in the ternary complexes comprise cognate nucleotides for at least two different base types.

23. The method of claim 22, wherein the nucleotides in the ternary complexes comprise cognate nucleotides for at least four different base types.

24. The method of any one of claims 1-23, further comprising detecting the ternary complexes.

25. The method of any one of claims 1-24, wherein the nucleotides comprise exogenous labels, and the method further comprises detecting the exogenous labels in the ternary complexes.

26. The method of claim 25, wherein detecting the exogenous labels in the ternary complexes comprises acquiring signals that distinguish at least two types of nucleotides in the ternary complexes.

27. The method of any one of claims 1-26, further comprising (c) contacting a plurality of second nucleotides with the plurality of polymerase-nucleic acid complexes after step (b), optionally in a vessel, thereby forming at least a second subset of the polymerase-nucleic acid complexes that comprises ternary complexes comprising second nucleotides from the plurality of second nucleotides.

28. The method of claim 27, wherein step (c) comprises delivering a solution comprising the plurality of second nucleotides to the vessel, thereby forming at least a second subset of the surface-immobilized polymerase-nucleic acid complexes that comprises ternary complexes comprising second nucleotides from the plurality of second nucleotides.

29. The method of any one of claims 27-28, wherein the second nucleotides comprise at least one different type of nucleotide compared to the nucleotides.

30. The method of any one of claims 27-28, wherein the second nucleotides comprise at least one same type of nucleotide compared to the nucleotides.

31. The method of any one of claims 27-30, further comprising detecting the ternary complexes that further comprise the second nucleotides.

32. The method of claim 31, wherein the second nucleotides comprise exogenous labels and the method further comprises detecting the exogenous labels in the ternary complexes that further comprise the second nucleotides.

33. The method of claim 32, wherein the ternary complexes that further comprise the second nucleotides are detected by acquiring signals that distinguish at least two types of the nucleotides.

34. The method of claim 32, wherein the ternary complexes that further comprise the second nucleotides are detected by acquiring signals that distinguish at least four types of the nucleotides.

35. The method of any one of claims 1-34, wherein the aqueous solution comprises a polyol.

36. The method of claim 35, wherein the polyol comprises polyethylene glycol or polyvinylalcohol.

37. The method of claim 35, wherein the polyol comprises a polymer.

38. The method of any one of claims 1-34, wherein the aqueous solution comprises an aliphatic diol.

39. The method of claim 38, wherein the aliphatic diol comprises a carbon chain that is non-branched.

40. The method of any one of claims 38-39, wherein the aliphatic diol comprises a carbon chain comprising seven or fewer carbons.

41. The method of claim 38, wherein the aliphatic diol is selected from the group consisting of propylene glycol, 1,3 butane diol, 1,5 pentanediol, 1,6 hexanediol and 1,7 heptanediol.

42. The method of any one of claims 1-34, wherein the aqueous solution comprises a sulfoxide.

43. The method of claim 42, wherein the sulfoxide is dimethyl sulfoxide.

44. The method of any one of claims 1-34, wherein the aqueous solution comprises a sulfone.

45. The method of claim 44, wherein the sulfone is ethyl methyl sulfone or sulfolane.

46. A method for identifying a nucleotide in a primed-template nucleic acid, comprising:
(a) providing a vessel comprising a primed-template nucleic acid, a polymerase and a nucleotide cognate of a first base type;
(b) examining the vessel for a stabilized ternary complex comprising the polymerase and the nucleotide cognate of the first base type bound at a base position of the primed-template nucleic acid;
(c) washing the vessel with an aqueous solution comprising a polyol, alcohol, aliphatic diol, sulfone, sulfoxide, or a combination thereof, thereby dissociating the nucleotide cognate of the first base type from the ternary complex, removing the nucleotide cognate of the first base type from the vessel and retaining the primed-template nucleic acid and the polymerase from step (b);
(d) delivering a nucleotide cognate of a second base type to the vessel after step (c);
(e) examining the vessel for a stabilized ternary complex comprising the polymerase and the nucleotide cognate of the second base type bound at the base position of the primed-template nucleic acid; and
(f) identifying the type of nucleotide at the base position of the primed-template nucleic acid.

47. The method of claim 46, wherein the aqueous solution further comprises Lithium, Betaine, or both.

48. The method of any one of claims 46-47, wherein the primed-template nucleic acid is surface-immobilized.

49. The method of claim 48, wherein the primed-template nucleic acid is one of a plurality of different surface-immobilized primed-template nucleic acids in an array, and wherein the method comprises identifying the type of nucleotide at a base position in each of the different surface-immobilized primed-template nucleic acids.

50. The method of any one of claims 46-49, wherein the vessel is selected from the group consisting of a flow cell, a well in a multi-well plate, a droplet, a vesicle, a test tube, a tray, a centrifuge tube, tubing and a channel in a substrate.

51. The method of any one of claims 46-50, wherein the nucleotide cognate of the first base type comprises an exogenous label and the nucleotide cognate of the second base type comprises an exogenous label.

52. The method of claim 51, wherein the exogenous label of the nucleotide cognate of the first base type is different from the exogenous label of the nucleotide cognate of the second base type.

53. The method of claim 52, wherein step (e) further comprises distinguishing signals from the different exogenous labels.

54. The method of claim 51, wherein the exogenous label on the nucleotide cognate of the first base type produces the same signal as the exogenous label on the nucleotide cognate of the second base type.

55. The method of any one of claims 46-54, further comprising
(g) delivering a nucleotide cognate of a third base type to the vessel, whereby the vessel retains the primed-template nucleic acid and the polymerase from step (b); and
(h) examining the vessel for a stabilized ternary complex comprising the polymerase and the nucleotide cognate of the third base type bound at the base position of the primed-template nucleic acid.

56. The method of claim 55, further comprising
(i) delivering a nucleotide cognate of a fourth base type to the vessel, whereby the vessel retains the primed-template nucleic acid and the polymerase from step (b); and
(j) examining the vessel for a stabilized ternary complex comprising the polymerase and the nucleotide cognate of the fourth base type bound at the base position of the primed-template nucleic acid.

57. The method of claim 56, further comprising
(k) adding a nucleotide to the primer of the primed-template nucleic acid, whereby the vessel comprises an extended primed-template nucleic acid;
(l) delivering a second polymerase and a nucleotide cognate of the first base type to the vessel; and
(m) repeating steps (b) through (i) using the extended primed-template instead of the primed-template nucleic acid and using the second polymerase instead of the polymerase.

58. The method of any one of claims 46-57, wherein the vessel of step (a) further comprises a nucleotide cognate of a third base type, and wherein step (b) comprises examining the vessel for a stabilized ternary complex comprising the polymerase and (i) the nucleotide cognate of the first base type bound at the base position of the primed-template nucleic acid or (ii) the nucleotide cognate of the third base type bound at the base position of the primed-template nucleic acid.

59. The method of claim 58, wherein the nucleotide cognate of the first base type comprises an exogenous label and the nucleotide cognate of the third base type comprises an exogenous label.

60. The method of claim 59, wherein the exogenous label of the nucleotide cognate of the first base type is different from the exogenous label of the nucleotide cognate of the third base type.

61. The method of claim 60, wherein step (b) further comprises distinguishing signals from the different exogenous labels.

62. The method of claim 59, wherein the exogenous label on the nucleotide cognate of the first base type produces the same signal as the exogenous label on the nucleotide cognate of the third base type.

63. The method of claim 62, wherein the examining of step (b) comprises detecting the signal.

64. The method of any one of claims 46-63, wherein step (c) further comprises delivering a nucleotide cognate of a fourth base type to the vessel, and wherein step (d) comprises examining the vessel for a stabilized ternary complex comprising the polymerase and (i) the nucleotide cognate of the second base type bound at the base position of the primed-template nucleic acid or (ii) the nucleotide cognate of the fourth base type bound at the base position of the primed-template nucleic acid.

65. The method of claim 64, wherein the nucleotide cognate of the second base type comprises an exogenous label and the nucleotide cognate of the fourth base type comprises an exogenous label.

66. The method of claim 65, wherein the exogenous label on the nucleotide cognate of the second base type is different from the exogenous label on the nucleotide cognate of the fourth base type.

67. The method of claim 66, wherein step (b) further comprises distinguishing signals from the different exogenous labels.

68. The method of claim 65, wherein the exogenous label on the nucleotide cognate of the second base type produces a signal that is not distinguished from the signal produced by the exogenous label on the nucleotide cognate of the fourth base type.

69. The method of claim 68, wherein the examining of step (b) comprises detecting the signal.

70. The method of any one of claims 46-69, further comprising
(g) adding a nucleotide to the primer of the primed-template nucleic acid, whereby the vessel comprises an extended primed-template nucleic acid;
(h) delivering a second polymerase and a nucleotide cognate of the first base type to the vessel; and
(i) repeating steps (b) through (e) using the extended primed-template instead of the primed-template nucleic acid and using the second polymerase instead of the polymerase.

71. The method of claim 70, wherein the polymerase and the second polymerase are the same type of polymerase.

72. The method of claim 70, wherein the primer comprises a reversible terminator moiety and wherein step (f) comprises deblocking the primer and adding the nucleotide to the deblocked primer, whereby the vessel comprises an extended primed-template nucleic acid.

73. The method of claim 72, wherein the nucleotide that is added to the primer comprises a reversible terminator moiety, whereby the extended primer comprises a reversible terminator moiety.

74. The method of any one of claims 46-73, wherein the aqueous solution comprises a polyol.

75. The method of claim 74, wherein the polyol comprises polyethylene glycol or polyvinylalcohol.

76. The method of claim 74, wherein the polyol comprises a polymer.

77. The method of any one of claims 46-73, wherein the aqueous solution comprises an aliphatic diol.

78. The method of claim 77, wherein the aliphatic diol comprises a carbon chain that is non-branched.

79. The method of any one of claims 77-78, wherein the aliphatic diol comprises a carbon chain comprising seven or fewer carbons.

80. The method of claim 77, wherein the aliphatic diol is selected from the group consisting of propylene glycol, 1,3 butane diol, 1,5 pentanediol, 1,6 hexanediol and 1,7 heptanediol.

81. The method of any one of claims 46-73, wherein the aqueous solution comprises a sulfoxide.

82. The method of claim 81, wherein the sulfoxide is dimethyl sulfoxide.

83. The method of any one of claims 46-73, wherein the aqueous solution comprises a sulfone.

84. The method of claim 83, wherein the sulfone is ethyl methyl sulfone or sulfolane.

85. A composition, comprising a plurality of polymerase-nucleic acid complexes in contact with an aqueous solution, wherein each of the plurality of polymerase-nucleic acid complex comprises a polymerase and a primed-template nucleic acid, wherein the aqueous solution comprises a polyol, alcohol, aliphatic diol, sulfone, sulfoxide, or a combination thereof.

86. The composition of claim 85, wherein at least a subset of the polymerase-nucleic acid complexes are ternary complexes further comprising nucleotides.

87. The composition of claims 85-86, wherein at most 50% of the polymerase-nucleic acid complexes are ternary complexes further comprising nucleotides.

88. The composition of claims 85-86, wherein at least 50% of the polymerase-nucleic acid complexes do not comprise nucleotides.

89. The composition of claims 85-88, wherein the aqueous solution further comprises Lithium, Betaine, or both.

90. The composition of any one of claims 85-89, wherein the aqueous solution comprises a polyol.

91. The composition of claim 90, wherein the polyol is selected from the group consisting of polyethylene glycol and polyvinyl alcohol.

92. The composition of claim 90, wherein the polyol comprises a polymer.

93. The composition of any one of claims 85-89, wherein the aqueous solution comprises an aliphatic diol.

94. The composition of claim 93, wherein the aliphatic diol comprises a carbon chain that is non-branched.

95. The composition of any one of claims 93-94, wherein the aliphatic diol comprises a carbon chain comprising seven or fewer carbons.

96. The composition of claim 93, wherein the aliphatic diol is selected from the group consisting of propylene glycol, 1,3 butane diol, 1,5 pentanediol, 1,6 hexanediol and 1,7 heptanediol.

97. The composition of any one of claims 85-89, wherein the aqueous solution comprises a sulfoxide, optionally the sulfoxide is dimethyl sulfoxide.

98. The composition of any one of claims 85-89, wherein the aqueous solution comprises a sulfone, optionally the sulfone is ethyl methyl sulfone or sulfolane.

99. The composition of any one of claims 85-98, wherein the polymerase-nucleic acid complex is surface-immobilized, and optionally the surface-immobilized polymerase-nucleic acid complex is covalently attached to the surface.

100. The composition of claim 99, wherein the polymerase of the surface-immobilized polymerase-nucleic acid complex, the primed-template nucleic acid of the surface-immobilized polymerase-nucleic acid complex, or both are covalently attached to the surface.

101. The composition of any one of claims 86-100, wherein the nucleotides comprise cognates for at least two different types of bases or comprises cognates for at least four different types of bases.

102. The composition of any one of claims 86-101, wherein one or more of the nucleotide, the polymerase, the primed-template nucleic acid comprises an exogenous label.
